(19) United States
(12) Patent Application Publication
(10) Pub. No.: US 2004/0033603 A1
(43) Pub. Date: Feb. 19, 2004

(54) Biotinylation of proteins

(76) Inventor: Lin Zhang, O'Fallon, MO (US)

Correspondence Address: Donald R. Holland, Harness, Dickey & Pierce, P.L.C., 7700 Bonhomme, Suite 400, Clayton, MO, 63105, US

(21) Appl. No.: 10/223,560

(22) Filed: Aug. 19, 2002

(51) Int. Cl. C07H 21/04; C12N 9/00; C12N 15/861; C12N 15/86

(52) U.S. Cl. 435/456; 435/320.1; 435/183; 536/23.2

(57) ABSTRACT

A eukaryotic bicistronic expression system for in vivo biotinylation of a protein is disclosed. The bicistronic expression system is based upon a polynucleotide which comprises a nucleic acid encoding a fusion protein made up of a selected protein and a biotinylation peptide, a nucleic acid sequence coding for an internal ribosome entry site and a nucleic acid sequence encoding a biotin ligase. Also disclosed are vectors and host cells containing the nucleic acid as well as methods for preparing a biotinylation protein and kits comprising the nucleic acid.

### BACKGROUND OF THE INVENTION

(1) Field of the Invention

This invention relates generally to the biotinylation of proteins, and, more particularly, to nucleic acid molecules, vectors and cells which can be used to produce biotinylated fusion proteins.

(2) Description of the Related Art

Modem biochemistry and cell biology depend heavily on the ability to covalently attach affinity tags to biomolecules such as proteins. An affinity tag, when used with a binding partner for the affinity tag, provides an aid to detection or isolation of a biomolecule.

One of the most commonly used affinity tags is the biotin moiety. Biotin, a naturally occurring vitamin, has several high-affinity binding partners, such as avidin, streptavidin, monomeric avidin, and anti-biotin antibodies (both polyclonal and monoclonal). The binding between biotin and avidin is among the strongest non-covalently attachments between molecules known, having a K_{d} of approximately 10⁻¹⁵ (Wilchek, M., and Bayer, E. A. (1988). The avidin-biotin complex in bioanalytical applications. Anal. Biochem. 171: 1-32). Because of this unusually high binding affinity, and because biotinylation of a biomolecule can often be accomplished without altering the protein's activity, biotinylation is one of the most important tools available for tagging proteins. For example, streptavidin immobilized on a solid support can be used to separate a biotin-tagged protein from a mixture: the biotinylated protein attaches to the immobilized streptavidin, and the mixture can be washed away, leaving the biotinylated protein attached to the solid support.

A commonly used procedure for covalently attaching a biotin moiety to a selected protein is to contact the protein with a chemically reactive derivative of biotin, for example sulfosuccinimidyl-biotin. When contacted with a protein presenting one or more primary amines, the sulfosuccinimidyl group of a sulfosuccinimidyl-biotin reacts with a primary amine on the protein, thereby covalently linking a biotin moiety to the protein. While this technology is robust, it is limited by the fact that a protein must be purified prior to its chemical biotinylation. In addition, chemical biotinylation has the disadvantages that the site(s) of biotinylation on the protein are often undefined or cannot be selected in advance, the number of biotins that attach to individual protein molecules vary, and the biotinylation can interfere with or destroy the protein's activity.

An alternative method for biotinylating proteins is to use enzymatic biotinylation methods. In particular, a biotin ligase enzyme can be used to attach a biotin moiety to a particular site within a specified polypeptide sequence. Biotin ligases (also known as biotin protein ligases) have been identified for numerous eukaryotic and prokaryotic species, for example E. coli, yeast, and human. Only a few endogenous proteins are known to be biotinylated and in some species only one such protein is known. The amino acid sequences that provide the biotinylation target sequence have been identified in certain proteins and the isolated sequence of amino acids constitutes a biotinylation peptide. Some of the identified biotinylation sequences have a minimum domain size of from 75 to 105 residues which are necessary for biotinylation to occur (Barker et al, J. Bol. Biol. 146:469-492, 1981; Eisenberg et al., J. Biol. Chem. 257:15167-15173, 1982). Using combinatorial approaches, shorter biotinylation peptides of 14-23 amino acids have been identified by Schatz and coworkers (Schatz, BioTechnology 11: 1138-1143, 1993; Beckett, et al., Protein Sci. 8: 921-929, 1999).

The development of in vivo biotinylation systems based upon enzymatic, site-specific biotinylation of target sequences have been reported. For example, Parrott and Barry have described a metabolic biotinylation system which uses recombinant mammalian cells (Parrott et al., Mol. Therap. 1:96-104, 2000). The cells were transformed with a plasmid encoding a fusion protein made up of a heterologous protein and a polypeptide corresponding to the biotin-acceptor domain of a known biotin acceptor protein. The minimal size of the biotin-acceptor domain was reported by this group to be 70 amino acids. Biotinylation resulted from the action of host cell biotin ligase. In a modification of this system, this group incorporated a second vector containing the E. Coli biotin ligase, Bir A, which was co-expressed with the fusion protein (Parrott et. al. Biochem. Biophys. Res. Comm. 281:993-1000, 2001).

Duffy et al. described the generation of a biotinylated fusion protein using a single vector encoding the fusion protein and a biotin ligase in a eukaryotic cell. (Duffy et al., Anal. Biochem. 262:122-128, 1998). The fusion protein was made up of a target protein and a biotin-acceptor peptide of 14 amino acids along with 6 additional amino acids. Enzymatic biotinylation was achieved in Spodoptera furugiperda cells by co-expression of both the fusion protein and E. coli Bir A enzyme from two separate promoters on a baculovirus vector.

Bicistronic expression of a fusion protein and a biotin ligase has also been reported in prokaryotic biotinylation systems. Tsao et al. reported on a plasmid vector which directed synthesis of recombinant proteins for biotinylation in E. coli. The protein of interest was produced as a fusion protein with a peptide substrate for E. coli Bir A enzyme. In one construction, the vector could also express the Bir A enzyme. Open reading frames for the various components terminated with a TAA codon and these were inserted between restriction enzyme sites XhoI and BamHI or the XmnI and BamHI. The BamHI site and the TAA stop codon were postulated to form a sequence (AGGA) that might serve as a prokaryotic internal ribosomal binding site.

Similarly, Skowronek et al. reported on a bicistronic expression unit for the expression of subunits of homodimeric proteins. The vector was constructed to express a fusion of a subunit protein followed by the coding sequence for a biotinylation peptide and this was followed by the coding sequence of Bir A. Potential prokaryotic ribosomal binding site sequences for translation initiation of the recombinant fusion protein (AAGAAG) and the Bir A enzyme (AAGGA) were identified in the plasmid sequence.

Both the Tsao et al. and the Skowronek et al. groups used prokaryotic biotinylation systems and neither of the groups suggested bicistronic expression in a eukaryotic system. This is not surprising, however, because, although prokaryotic cells commonly express proteins bicistronically, eukaryotic cells do not. Indeed, eukaryotic mRNA is predomantly translated in a monocistronic manner. As a result, biotinylation in eukaryotic bicistronic expression systems has not been reported or suggested heretofore.

### BRIEF SUMMARY OF THE INVENTION

Accordingly, the inventors herein have succeeded in developing a eukaryotic biotinylation system in which a nucleic acid sequence encoding a biotin ligase enzyme and a fusion protein are expressed bicistronically. The fusion protein comprises a selected protein and a biotinylation peptide which can be biotinylated by the biotin ligase enzyme. Bicistronic expression results from the placement of the coding sequence for an internal ribosomal entry site 3′ to the first coding sequence which is, preferably, the sequence encoding the fusion protein and immediately 5′ to the second coding sequence which, preferably, encodes the biotin ligase molecule. The polynucleotide containing the coding sequences can be an RNA molecule or a DNA molecule in which the polynucleotide is operatively linked to a eukaryotic promoter operatively linked to the polynucleotide. As such, an RNA transcript is produced in a eukaryotic cell or in an in vitro system derived from a eukaryotic cell. The resultant RNA transcript allows for bicistronic translation of both the fusion protein and the biotin ligase. Co-expression of the fusion protein and the biotin ligase from the same transcript provides both enzyme and substrate such that a biotinylated fusion protein is efficiently generated. Bicistronic expression in mammalian cells also allows for the production of functional proteins, in particular, proteins which require complex folding or post-translational modifications which is often not readily achieved upon expression from prokaryotic cells.

Thus, the present invention is directed, in general, to a nucleic acid molecule comprising a polynucleotide which encodes a fusion protein, an internal ribosome entry site and a biotin ligase. The fusion protein comprises a selected protein and a biotinylation peptide capable of being biotinylated by the biotin ligase. In one embodiment, the nucleic acid molecule is a DNA molecule comprising a eukaryotic promoter operatively linked to the polynucleotide. Transcription of the DNA molecule provides an mRNA sequence encoding the fusion protein, an internal ribosome entry site and an RNA sequence encoding the biotin ligase. Translation of the mRNA in a eukaryotic cell or in an in vitro translation system derived from eukaryotic cells produces both the fusion protein and the biotin ligase enzyme. The enzymatic activity of the biotin ligase catalyzes the biotinylation of the fusion protein.

In another embodiment, the polynucleotide initially does not contain the coding sequence for the selected protein, but, instead, the polynucleotide contains a site allowing for insertion of a DNA sequence encoding the selected protein. The polynucleotide also contains coding sequence for a biotinylation peptide, an IRES sequence and coding sequence for Bir A. The DNA sequence encoding a selected protein (either genomic DNA or a cDNA) is then inserted into the polynucleotide in the same reading frame as the biotinylation peptide, such that expression of the DNA molecule yields the protein of interest fused to the biotinylation peptide. The fusion protein becomes biotinylated by the biotin ligase expressed from the same transcript.

Preferably, the site allowing for insertion of a DNA sequence comprises one or more restriction enzyme cleavage sites (e.g., a polylinker) or a recombination site or a topisomerase I-based site. The recombination site is a DNA sequence subject to site specific recombination, for example a bacteriophage lambda att site.

In another embodiment, the polynucleotide is a DNA cassette for generating a DNA molecule encoding a fusion protein comprising a selected protein and a biotinylation peptide, the cassette comprising a DNA sequence encoding a biotinylation peptide, a DNA sequence coding for an internal ribosome entry site, and a DNA sequence encoding a biotin ligase. The cassette is suitable for insertion into a construct comprising a eukaryotic promoter operatively linked to coding sequence for a selected protein (either genomic sequence or cDNA sequence). The cassette is inserted such that biotinylation peptide is in the same reading frame as the coding sequence for the selected protein and, preferably, 3′ and immediately adjacent to the selected protein. The construct is expressed in a eukaryotic cell or in an in vitro expression system with subsequent biotinylation leads to formation of a fusion peptide comprising the selected protein and the biotinylation peptide, as well as the biotin ligase. The cassette can be inserted by any standard technique such as restriction endonuclease digestion, recombination, or topoisomerase I-based cloning.

In another embodiment, the polynucleotide is an RNA sequence encoding a fusion protein and an RNA sequence encoding a biotin ligase operatively linked to an internal ribosome entry site. The fusion protein comprises a selected protein and a biotinylation peptide capable of being biotinylated by the biotin ligase. Translation of the RNA in a eukaryotic environment yields the fusion protein and the biotin ligase which subsequently generates a biotinylated fusion protein.

The present invention is also directed to vectors and host cells containing the nucleic acid molecules of the present invention, to kits containing the nucleic acid molecules and/or vectors and/or cells of the present invention and to methods of preparing a biotinylated fusion protein using the nucleic acid constructs of the present invention either in vivo in a host cell or in vitro.

### BRIEF DESCRIPTION OF THE DRAWINGS

FIG. 1 illustrates bicistronic expression vectors, (A) pFLAG-p53-BP and (B) pFLAG-p53-BP.

FIG. 2 illustrates Western blot analysis of recombinant p53 fusion protein expressed from COS-7 cells transfected with a control vector (lane 1), with pFLAG-p53-BP (lane 2) and with pFLAG-p53-BP-bir A (lane 3) probed with anti-FLAG M2 HRP conjugate (panel A) or streptavidin-HRP conjugate (panel B).

FIG. 3 illustrates the immobilization of biotinylated FLAG-p53-BP fusion protein on streptavidin plates detected using anti-FLAG-M2 HP conjugate showing optical density at 450 nm of lysates of (●) cells transfected with empty vector, (∘) cells transfected with pFLAG-p53-BP and (▾) cells transfected with pFLAG-p53-BP-bir A.

FIG. 4 illustrates (A) the lack of effect of increasing biotin concentration in media for cells transfected with empty vector (black bar), cells transfected with pFLAG-p53-BP (light gray bar) and cells transfected with pFLAG-p53-BP-bir A and (B) Western blot probed with anti-FLAG M2 HRP conjugate, showing expression of FLAG-p53-BP fusion protein from cells transfected with pFLAG-p53-BP-bir A and cultured at three different concentrations of biotin.

FIG. 5 illustrates the maintained functionality of the fusion protein, FLAG-p53-BP, by detection of the interaction of large T-antigen and FLAG-p53-BP co-precipitated on anti-FLAG M2 plate, as shown in an ELISA analysis using anti-SV40 Large T and small T antibody for cells transfected with empty vector (●), cells transfected with pFLAG-p53-BP (▾), and cells transfected with pFLAG-p53-BP-bir A (▪).

FIG. 6 illustrates the interaction between biotinylated FLAG-p53-BP and exogenous c-Myc-large T-antigen co-precipitated on a streptavidin plate as shown in an ELISA analysis using anti-SV40 Large T and small T antibody for cells transfected with empty vector (●), and cells transfected with pFLAG-p53-BP-bir A (▪).

FIG. 7 illustrates the detection of intracellular biotinylated FLAG-p53-BP in COS-7 cells transfected with pFLAG-p53-BP bir A using dual fluorescence labeling with FITC-conjugated anti-FLAG M2 antibody and Cys-conjugated streptavidin showing the transfected COS-7 cell labeling with (A) FITC, (B) Cy3 (C) FITC and Cy3 co-localized and (D) the FITC and Cy3 co-localized labeling image in C enlarged.

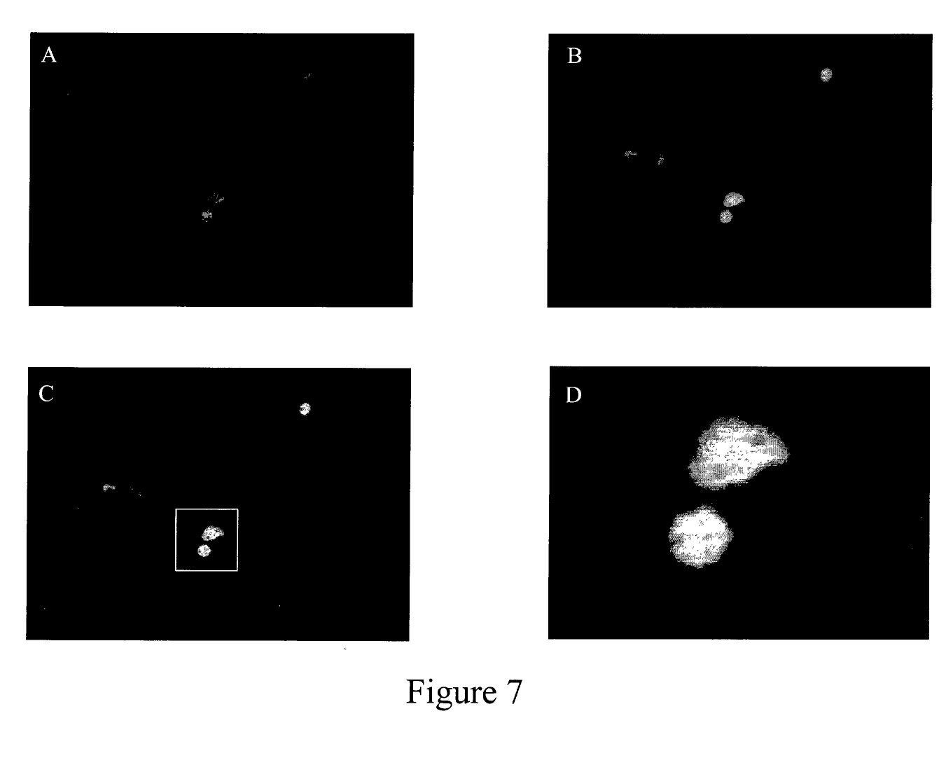

### DETAILED DESCRIPTION OF THE INVENTION

In accordance with the present invention, it has been discovered that biotinylated fusion proteins can be produced using a eukaryotic bicistronic expression system in vivo or in vitro. The system expresses a biotin ligase molecule and a fusion protein capable of being biotinylated by the biotin ligase molecule. The fusion protein is made up of a selected protein and a short biotinylation peptide which is capable of being biotinylated by the biotin ligase molecule. The bicistronic expression system is comprised of a polynucleotide which contains the coding sequences of the fusion protein and the biotin ligase. Preferably, the polynucleotide contains from 5′ to 3′, the coding sequence for the fusion protein, the coding sequence for an internal ribosome entry site and the coding sequence to a biotin ligase molecule. The polynucleotide can be a DNA molecule or an RNA molecule which is operatively linked to a eukaryotic or bacteriophage promoter such that an RNA transcript is either produced in a eukaryotic cell or in an in vitro system derived from a eukaryotic cell respectively. The resultant mRNA transcript allows for translation of both the fusion protein and the biotin ligase.

The bicistronic expression systems of the present invention can be used to biotinylate a wide variety of proteins while retaining the functional activity of the protein in the biotinylated form. Because the expression system is a eukaryotic expression system, complex eukaryotic proteins can be produced with proper folding and/or post-translational modifications such that the proteins are generated in their functionally active forms. In addition, the biotinylation peptide portion of the fusion protein is a short polypeptide, which can be as short as 14 or 15 amino acid and up to about 65 to about 70 amino acids in length. As a result the biotinylation peptide would not be expected to interfere with the functional activity of the selected protein to which it is fused.

Non-limiting examples of some of the proteins which can be biotinylated using the present bicistronic system include for illustrative purposes only, hormones, including thyroid-stimulating hormone, follicle-stimulating hormone, luteinizing hormone, prolactin, growth hormone, adrenocorticotropic hormone, growth hormone-releasing hormone, corticotropin-releasing hormone, somatostatin, calcitonin, parathyroid hormone, human chorionic gonadotropin, insulin, glucagon, somatostatin, erythropoietin, atrial-natriuretic peptide, gastrin, secretin, cholecystokinin, somatostatin, neuropeptides, insulin-like growth factor-1, angiotensinogen, thrombopoietin and leptin; enzymes, including oxidoreductases including dehydrogenases, oxidases, reductases and catalases; transferases including acetyltransferases, methylases, protein kinases and phosphatases; hydrolases including proteases, nucleases and phosphatases; lyases including decarboxylases and aldolases; isomerases, including epimerases and racemases; and ligases including peptide synthases, aminoacyl-tRNA synthetases, DNA ligases and RNA ligases; cell surface proteins such as transport proteins and receptor proteins; intracellular proteins such as proteins associated with intracellular signaling such as G-proteins and associated receptors, proteins associated with intracellular transport, structural proteins, and numerous other proteins.

The second component of the fusion protein is the biotinylation peptide. Preferably, the biotinylation peptide is at least a 14-mer and preferably a 15-mer sequence. Short biotinylation peptides have been identified based upon a random peptide generating and screening system to generate substrate peptides for the E. coli biotin ligase enzyme, Bir A (see Schatz, Biotechnology 11:1 138-1142, 1993; U.S. Pat. No. 5,723,584). A consensus sequence was identified to be Leu-Xaa₁-Xaa₂, Ile-Xaa₃-Xaa₄-Xaa₅-Xaa₆-Lys-Xaa₇-Xaa₈-Xaa₉-Xaa₁₀, where Xaa₁ is any amino acid; Xaa₂ is any amino acid other than Leu, Val, Ile, Trp, Phe, or Tyr; Xaa₃ is Phe or Leu; Xaa₄ is Glu or Asp; Xaa₅ is Ala, Gly, Ser, or Thr; Xaa₆ is Gln or Met; Xaa₇ is Ile, Met, or Val; Xaa₈ is Glu, Leu, Val, Tyr, or Ile; Xaa₉ is Trp, Tyr, Val, Phe, Leu, or Ile; and Xaa₁₀ is any amino acid other than Asp or Glu (SEQ ID NO: 1).

The random peptide generating system, however, was biased toward the expression of peptides corresponding to earlier known sequences of several biotinylated proteins (Samols et al., J. Biol. Chem. 263:6461-6464, 1988). For example, the −1 and +1 positions relative to the Lysine residue that becomes biotinylated are biased toward Met with the variation in the −1 position identified as Gin in addition to Met and the +1 position identified as Ile or Val in addition to Met. Nevertheless, additional variants can be biotinylated. For example, Shenoy et al. showed that biotinylation takes place in mutant sequences in which the −1 position (Xaa₆ in the consensus sequence) is Leucine or Threonine and the +1 position (Xaa₇ in the consensus sequence) is Leucine. These mutant variations are, however outside of the consensus sequence identified in U.S. Pat. No. 5,723,584, which indicates that a larger number of biotinylation peptides can serve as substrates for a biotin ligase enzyme, in particular Bir A than is represented by the consensus sequence. Variations in the consensus sequence can be constructed in which the conservative amino acid substitutions are made in the consensus sequence with amino acids having similar characteristics such as size, charge, acid/base character and hydrophobicity of the amino acid. For example, in the −4 position of the consensus sequence of U.S. Pat. No. 5,723,584 Phe or Leu are indicated, however, Ile is of a similar size and hydrophobicity as Leu (see for example, Black et al, Anal. Biochem. 193:72-82, 1991) so that a broader range of amino acids for the −4 position is Phe, Leu or Ile. For the −3 position, the consensus sequence identifies Glu or Asp, however, Arg and Thr are shown to be present at that position in SEQ ID NO: 15 and SEQ ID NO: 28 of U.S. Pat. No. 5,723,584.

Thus, a number of variations in the consensus sequence disclosed in U.S. Pat. No. 5,723,584 have been identified herein. The skilled artisan will appreciate that further variations are also possible. As such, a broader range of biotinylation peptides than represented by the consensus sequence of U.S. Pat. No. 5,723,584 are possible.

The above-identified, as well as additional variations in the consensus sequence constitute conservative amino acid substitutions. A conservative amino acid substitution refers to the interchangeability of one amino acid for another having similar chemical properties as a result of having similar side chains. For example, alanine, valine, leucine, isoleucine, proline, tryptophan, phenylalanine and methionine are all neutral and hydrophobic amino acids; glycine, serine, threonine, tyrosine, cysteine, asparagine and glutamine are all neutral and polar amino acids; lysine, arginine and histidine are all basic; aspartic acid and glutamic acid are acidic amino acids; glycine, alanine, valine, leucine, and isoleucine all have aliphatic side chains; serine and threonine have aliphatic-hydroxyl side chains; asparagine and glutamine have amide-containing side chains; phenylalanine, tyrosine, and tryptophan all have aromatic side chains; and cysteine and methionine have sulfur-containing side chains. Preferred conservative amino acids substitution groups are: valine-leucine-isoleucine, phenylalanine-tyrosine, lysine-arginine, alanine-valine, asparagine-glutamine, glutamic acid-aspartic acid, leucine-methionine, and glutamine-histidine. Other biotinylation peptides have also been described (see for example, Samols et al., J. Biol. Chem. 263:6461-6464, 1988; Murtif et al., Proc. Natl. Acad. Sci. U.S.A. 82: 5617-5621 (1985)). These can also be used in the present invention by fusing to the selected peptide so long as the functional activity of the selected peptide is not substantially diminished.

A preferred sequence which includes a 13 amino acid sequence of the consensus sequence with the additional amino acids is the 17 amino acid sequence Gly-Ser-Gly-Leu-Asn-Asp-Ile-Xaa₁-Xaa₂-Ala-Xaa₃-Lys-Xaa₃-Glu-Trp-His-Glu where Xaa₁ is Phe, Leu or Ile, Xaa₂ is Glu-Asp, Arg or Thr, Xaa₃ is Gln, Met, Leu or Thr, and Xaa₃ is Ile, Met, Val, Leu, Tyr, or Trp (SEQ ID NO: 2). One preferred biotinylation peptide sequence is Gly-Ser-Gly-Leu-Asn-Asp-Ile-Phe-Glu-Ala-Gln-Lys-Ile-Glu-Trp-His-Glu (SEQ ID NO: 3).

The biotinylation peptide can be fused to the selected protein at either the amino or carboxy terminus, but preferably at the carboxy terminus. Additional amino acids can also be added to the sequence. For example, if the biotinylation peptide is fused to the amino terminus, a methionine can be added as the first amino acid to facilitate initiation of translation of the nucleic acid encoding the fusion protein. Additional amino acids can also be used to serve as linkers to the selected protein.

The biotin ligase enzyme is another component of the biotinylation system. Biotin ligase enzymes or biotin protein ligase enzymes as referenced herein are intended to include any of the prokaryotic or eukaryotic enzymes which catalyze the covalent attachment of biotin to a polypeptide via an amide linkage between the biotin carboxyl group and the amino group of a lysine of the polypeptide. Biotin ligase enzymes from a number of species are known in the art (see for example, Barker et al. J. Mol. Biol., 146:468-492,1981; Bower et al, J. Bacteriol 177:2572-2575, 1995; Chapman-Smith et al. J. Nutr. 129:477S-484S, 1999; Cronan et al, Methods in Enzymology 326:440-458, 2000). The amino acid sequences as well as nucleic acid sequences encoding a number of the enzyme have been identified in annotated genomic sequences (see, for example, National Center for Biotechnology Information, Entrez-Nucleotide database). Any biotin ligase can thus be use in the present invention so long as the enzyme is capable of biotinylating the biotinylation peptide portion of the fusion protein. A preferred biotin ligase is the E. coli biotin ligase Bir A (see Barker et al. supra, 1981; Howard et al., Gene 35:321-331, 1985).

Another component of the bicistronic expression system of the present invention is the internal ribosome entry site. Whereas the first protein of the bicistronic expression system will be typically translated by a 5′ end-dependent mechanism, with a 5′ terminal cap structure and methionine initiation codon, the internal ribosome entry site allows the bicistronic expression of the second protein coding sequence by an cap- and end-independent mechanism. Either of the fusion protein or the biotin ligase enzyme coding sequences can be the second protein, i.e. 3′ to the other, and initiation of translation of the second coding sequence is commenced as a result of an operative linkage of that coding sequence to the internal ribosome entry site. Preferably, the biotin ligase enzyme coding sequence is the second coding sequence whose translation is initiated by the internal ribosome entry site.

Any of a number of internal ribosomal entry sites are known and suitable for use in the bicistronic expression system of the present invention. A number of internal ribosome entry sites have been identified, including those of RNA Picornaviridae and in some instances, DNA viridae, as well as cellular mRNA's (Hellen et al., Genes & Develop. 15:1593-1612, 2001). Internal ribosome entry site elements have nucleotide lengths of from a few nucleotides such as about 130 to long chains of nucleotides of about 460 nucleotides or greater and although the sequences differ as do their secondary structures, from a functional point of view, all perform the same function (Martinex-Salas, Current Opinion in Biotechnology 10:458-464, 1999). Particularly preferred is the encephalomyocarditis internal ribosome entry site as disclosed in U.S. Pat. No. 4,937,190 (ATCC 67525) (see also Rees et al., BioTechniques 20:102-110, 1996; Jackson et al., Trends Biochem. Sci. 15:477-483, 1990; Jang et al, J. Virol. 62:2636-2643, 1990). The DNA sequence is as follows:

The DNA sequence is as follows:

(SEQ ID NO: 4)

AATTCCGCCCCTCTCCCTCCCCCCCCCCTAACGTTACTGGCCGAAGCCGC
TTGGAATAAGGCCGGTGTGCGTTTGTCTATATGTGATTTTCCACCATATT
GCCGTCTTTTGGCAATGTGAGGGCCCGGAAACCTGGCCCTGTCTTCTTGA
CGAGCATTCCTAGGGGTCTTTCCCCTCTCGCCAAAGGAATGCAAGGTCTG
TTGAATGTCGTGAAGGAAGCAGTTCCTCTGGAAGCTTCTTGAAGACAAAC
AACGTCTGTAGCGACCCTTTGCAGGCAGCGGAACCCCCCACCTGGCGACA
GGTGCCTCTGCGGCCAAAAGCCACGTGTATAAGATACACCTGCAAAGGCG
GCACAACCCCAGTGCCACGTTGTGAGTTGGATAGTTGTGGAAAGAGTCAA
ATGGCTCTCCTCAAGCGTATTCAACAAGGGGCTGAAGGATGCCCAGAAGG
TACCCCATTGTATGGGATCTGATCTGGGGCCTCGGTGCACATGCTTTACA
TGTGTTTAGTCGAGGTTAAAAAAACGTCTAGGCCCCCCGAACCACGGGGA
CGTGGTTTTCCTTTGAAAAACACGATGATAA,
and the RNA sequence is as follows:

(SEQ ID NO: 5)
AAUUCCGCCCCUCUCCCUCCCCCCCCCCUAACGUUACUGGCCGAAGCCGC
UUGGAAUAAGGCCGGUGUGCGUUUGUCUAUAUGUGAUUUUCCACCAUAUU
GCCGUCUUUUGGCAAUGUGAGGGCCCGGAAACCUGGCCCUGUCUUCUUGA
CGAGCAUUCCUAGGGGUCUUUCCCCUCUCGCCAAAGGAAUGCAAGGUCUG
UUGAAUGUCGUGAAGGAAGCAGUUCCUCUGGAAGCUUCUUGAAGACAAAC
AACGUCUGUAGCGACCCUUUGCAGGCAGCGGAACCCCCCACCUGGCGACA
GGUGCCUCUGCGGCCAAAAGCCACGUGUAUAAGAUACACCUGCAAAGGCG
GCACAACCCCAGUGCCACGUUGUGAGUUGGAUAGUUGUGGAAAGAGUCAA
AUGGCUCUCCUCAAGCGUAUUCAACAAGGGGCUGAAGGAUGCCCAGAAGG
UACCCCAUUGUAUGGGAUCUGAUCUGGGGCCUCGGUGCACAUGCUUUACA
UGUGUUUAGUCGAGGUUAAAAAAACGUCUAGGCCCCCCGAACCACGGGGA
CGUGGUUUUCCUUUGAAAAACACGAUGAUAA.

The bicistronic expression system, in one embodiment involves a recombinant DNA molecule which is expressed in a eukaryotic cell. The DNA molecule, in such embodiments is present in an expression vector which comprises a eukaryotic promoter operatively linked to the polynucleotide encoding the fusion protein, the internal ribosome entry site and the biotin ligase. The promoter can be any eukaryotic or bacteriphage promoter that will direct transcription in the eukaryotic host cell or in an in vitro expression system derived from the eukaryotic host cell respectively. The promoter can be of cellular or, more preferably of viral origin. One such promoter is the human cytomegalovirus immediate early promoter.

Additional components of the bicistronic expression system can be included in the DNA molecule to enhance transcription or translation levels, such as for example, enhancers, operators, start signals, stop signals, cap signals, polyadenylation signals, and the like.

A 5′ leader sequence can also be present which encodes a signal peptide such that the full sequence encodes a preprotein for membrane binding and secretion of the protein. A pro sequence can also be present to allow for generation of a stable precursor of the protein. Moreover, a prepro region can be present to encode a prepro-protein in which the pre sequence facilitates secretion and is cleaved during the secretion process whereas the pro-protein forms a stable substrate for later cleavage to form the functionally active protein.

The leader peptide when present is contiguous with the amino terminal of the fusion protein. The leader peptide directs a newly synthesized protein to the secretion pathway of a cell. The presence of a leader peptide leads to secretion of the fusion protein into the medium by cells expressing the fusion protein. The presence of the leader sequence provides the benefit that the fusion protein can be collected from cell culture medium, obviating the need to form a cell lysate to obtain the fusion protein. The leader peptide sequence can be from any secreted protein that uses a leader peptide for secretion. The leader peptide sequence is preferably that of an immunoglobulin, more preferably that of a mouse immunoglobulin, and most preferentially that of a mouse IgG.

The bicistronic expression of the fusion protein and the biotin ligase are similar, but need not be identical. Expression should be efficient such that the fusion peptide is efficiently biotinylated.

The bicistronic expression system of the present invention can be in certain embodiments the form of a cassette or a kit containing the cassette. The cassette is a DNA molecule constructed for insertion of a DNA sequence encoding a selected protein. The DNA molecule is comprised of a eukaryotic promoter operatively linked to a polynucleotide which comprises a site allowing for insertion of the DNA molecule encoding the selected protein, a DNA sequence encoding a biotinylation peptide appropriately positioned to form a coding sequence for a fusion protein of the selected protein fused to the biotinylation protein, a DNA sequence coding for an internal ribosome entry site, and a DNA sequence encoding a biotin ligase. The cassette allows for convenient insertion a DNA sequence encoding a selected protein into the bicistronic expression system for the production of a biotinylated fusion protein.

Any method of insertion can be used, provided that the DNA encoding the selected protein and the DNA encoding the biotinylation peptide are in the same reading frame, and no translation termination codon intervenes between the two coding sequences. For example, the site allowing for insertion can comprise a restriction enzyme cleavage site. When the site is cleaved using a restriction enzyme, a DNA such as an amplicon generated using the polymerase chain reaction (PCR), can be inserted “in frame,” thus yielding a sequence encoding a fusion protein. Alternatively, the site allowing for insertion can be a recombination site. The recombination site can be from any available source, and is preferably a bacteriophage lambda att site. Alternatively, a DNA such as an amplicon can be inserted using topoisomerase I-based sites.

Kits are also within the scope of the present invention. The kits can contain the DNA cassettes packaged in a container. Along with the DNA cassettes, such kits can also contain appropriate reagents for use in insertion of the DNA sequence encoding the selected protein into the bicistronic expression cassettes. In addition, an instruction manual can be included for inserting the selected protein into the cassettes.

In another embodiment, the DNA molecule of present invention can be in the form of a DNA cassette for insertion into a pre-existing construct. For example, the pre-existing construct can be a plasmid comprising a eukaryotic promoter operatively linked to a cDNA encoding a selected protein. Upon insertion of the cassette, the resulting plasmid comprises a eukaryotic promoter operatively linked to a polynucleotide comprising a DNA sequence encoding a fusion protein comprising the selected protein and a biotinylation peptide, a DNA sequence coding for an internal ribosome entry site, and a DNA sequence encoding a biotin ligase. The insertion can be accomplished by any of a number of standard methods used in molecular biology (see Sambrook, J. C., Fritsch, E. F., and Maniatis, T. (1989) Molecular Cloning: A Laboratory Manual, Cold Spring Harbor Laboratory Press, Cold Spring Harbor, N.Y.).

Kits which contain the DNA cassettes for insertion into a pre-existing construct are also within the present invention. Such kits can contain the DNA cassettes packaged in a container. Along with the DNA cassettes, the kits can also contain appropriate reagents for use in insertion of the DNA cassette into a pre-existing construct to produce a bicistronic expression vector. In addition, an instruction manual can be included for inserting the cassette into the pre-existing construct.

The nucleic acid of the present invention can also be an RNA molecule. The RNA molecule comprises an RNA sequence encoding a fusion protein comprising a selected protein and a biotinylation peptide; and an RNA sequence encoding a biotin ligase operatively linked to an internal ribosome entry site. The RNA can be produced preferably in an in vitro system derived from a eukaryotic cell system. Preferably, the RNA is produced by in vitro transcription using a bacteriophage RNA polymerase from phages T7, T3, or SP6 in which a corresponding DNA molecule forms the template. The DNA template comprises a T7, T3 or SP6 promoter operatively linked to polynucleotide comprising a DNA sequence encoding a fusion protein of a selected protein fused to a biotinylation peptide, a DNA sequence coding for an internal ribosome entry site, and a DNA sequence encoding a biotin ligase. To facilitate translation of the upstream coding sequence in a eukaryotic cell, a m7G(5′)ppp(5 ′)G cap is introduced to in vitro transcription reaction mixtures.

The nucleic acids of the present invention can also include a sequence encoding a protease cleavage site which allows the selected protein to be cleaved from the fusion protein. The protease cleavage site is thus located between the selected protein and the biotinylation peptide. The cleavage site can be used to separate the selected protein from the biotinylation peptide bound to a solid support. For example, a liquid mixture containing a biotinylated fusion protein which is made up of the selected protein, a protease cleavage site, and a biotinylation peptide can be contacted with an immobilized biotin binding partner. One such example of an immobilized biotin binding partner is streptavidin attached to plastic beads. Following contact with the beads, the mixture can be washed away, leaving the biotinylated fusion protein attached to the beads. The selected protein can be then released into solution from the beads by contacting the beads with a protease that selectively cleaves the fusion protein at the protease recognition site. This procedure yields highly purified selected protein.

The protease is preferably an enterokinase (EK), and the protease cleavage site is the EK recognition site comprising the amino acid sequence Asp-Asp-Asp-Asp-Lys (SEQ ID NO: 6). EK cleaves the fusion protein after the C-terminal lysine of the recognition site.

In the embodiments of the present invention that comprise a DNA molecule, the DNA molecule can be single stranded or double stranded. Preferably, the DNA is double stranded. The DNA molecule can be an amplicon, a linear DNA molecule, or a circular DNA molecule. Preferably, the circular DNA molecule comprises a plasmid. The DNA can also be a recombinant virus or artificial chromosome.

The host cell of the present invention is a eukaryotic cell that supports translation initiated both from the 5′ end of a transcript and from an internal ribosome entry site. Non-limiting examples include mammalian cells, frog oocytes, yeast cells, and plant cells. Preferably, the host cell is a mammalian cell. More preferably, the host cell is a COS cell.

The nucleic acids of the present invention are introduced into cells by standard techniques, for example infection, transfection, or injection. Only a single species of a nucleic acid needs to be introduced to the cells. The cells are grown or maintained by standard techniques. Expression of recombinant DNA or RNA leads to production of both the fusion protein and the biotin ligase, whereby the fusion protein becomes biotinylated by the biotin ligase.

The fusion protein of the present invention can, in certain embodiments, also comprise an epitope tag in addition to the biotinylation site. “Epitope tag” means any site on a molecule for which a binding partner is available, and includes but is not limited to epitopes recognized by antibody molecules, but specifically excludes biotin. Preferably, the epitope tag is a FLAG sequence (comprising the sequence Asp-Tyr-Lys-Asp-Asp-Asp-Asp-Lys (SEQ ID NO: 7), a myc epitope tag, or a polyhistidine tag. Most preferably, the epitope tag is a FLAG sequence. The epitope tag provides a binding site that can be used for purification, detection, identification, localization, or protein-protein interaction studies. For example, to isolate a fusion protein comprising a polyhistidine tag from a mixture, the mixture is contacted with a nickel ion column. The column is then washed, and substantially pure fusion protein is then eluted from the column by contacting the high concentration of imidazole. In another example, a fusion protein comprising a FLAG sequence can be localized in a cell or tissue by exposing the cell or tissue to an anti-FLAG sequence antibody as a probe. A tag on the anti-FLAG antibody, such as a fluorophore or a secondary antibody tagged with a fluorophore, is used to visualize the presence and distribution of the fusion protein. In addition, because the FLAG sequence comprises the EK cleavage sequence, in fusion proteins comprising a FLAG sequence, the selected protein can be separated from the biotinylation peptide by cleavage with enterokinase. The skilled artisan will also, readily, appreciate that the epitope-tagged protein can also be used in Western blotting, reporter assays, ELISA assays, protein-protein interaction assays and the like.

The present invention is also directed to production of biotinylated fusion protein in vitro. The nucleic acids of the invention encoding a fusion protein can be transcribed and/or translated in any suitable eukaryotic expression system, for example a rabbit reticulocyte lysate coupled transcription/translation system, a wheat germ extract translation system, or a Xenopus oocyte extract translation system. In addition, transcription can also be effected in vitro using standard methods and techniques. For example, a bacteriophage T3, T7, or SP6 promoter is incorporated into the DNA molecule, and used in combination with the corresponding RNA polymerase to direct transcription in vitro. Furthermore, a m7G(5′)ppp(5 ′)G cap can also be included in the synthesis of a transcript. The synthetic transcript is then added to an in vitro translation system for efficient translation of both the fusion protein and the biotin ligase.

Illustrative examples are described below. Unless otherwise indicated, all materials used in the examples were obtained from Sigma-Aldrich Corporation, St. Louis, Mo.

### EXAMPLE 1

This example illustrates the construction of bicistronic expression vectors pFLAG-p53-BP and pFLAG-p53-BP-bir A and the monocistronic expression vector pc-Myc-CMV-2-large T-antigen and cell transfection.

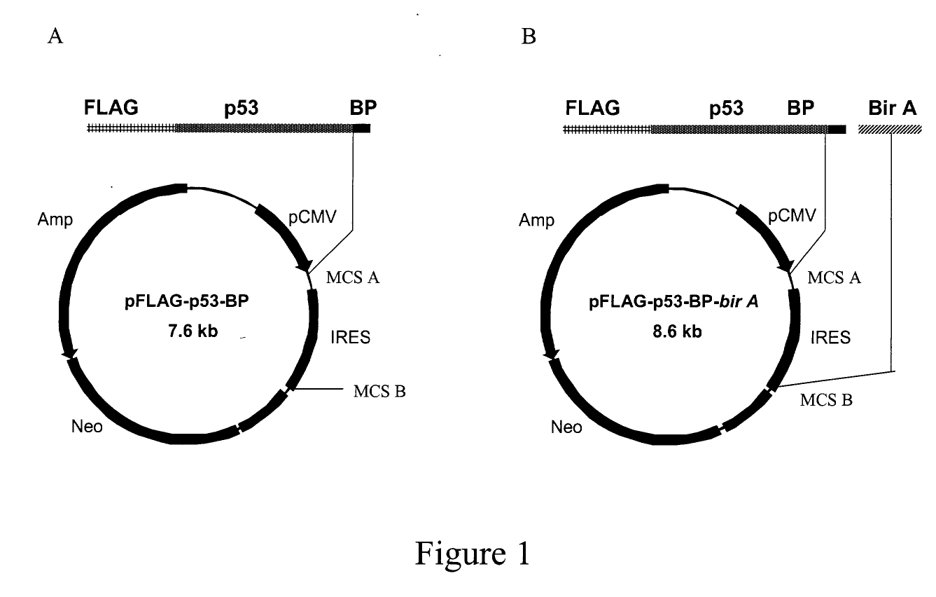

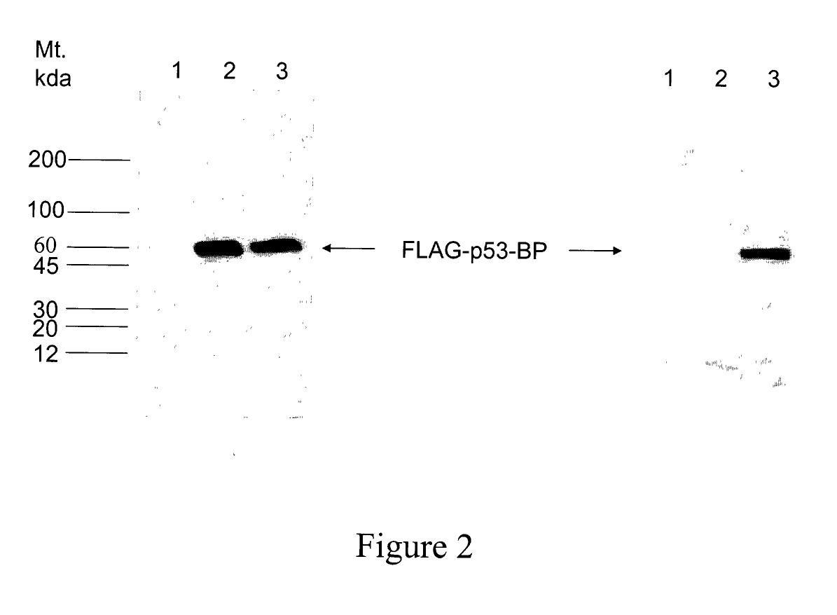

p53 was selected as the model protein for the evaluation of the suitability of the BP as a substrate for E. coli Bir A biotinylation in mammalian cells. p53 is a tumor suppressor protein originally identified as a protein bound to large T-antigen in SV40-transformed cells (Lane et al. Nature (London) 278:261-263, 1979; Linzer et al. Cell 17:43-52, 1979; Jenkins et al. J. Virol. 62:3903-3906, 1988; Gluzman et al. Cell 23:175-82, 1981; Wang et al., Cell 57:379-392, 1989). Two bicistronic expressing vectors were constructed expressing recombinant FLAG-p53-BP. One expression vector, pFLAG-p53-BP (FIG. 1 left), encodes a dual-tagged p53 fusion protein whose N-terminus was tagged with a FLAG sequence (FLAG) and a 17 residue BP sequence (BP) at the C-terminus. The BP sequence is organized as two amino acid residues (GS) in front of a 15-mer sequence (GLNDIFEAQKIEWHE) (SEQ ID NO: 8) (Avidity, Denver, Colo.). The utility of this vector allows us to determine whether the endogenous biotinylation enzymes in mammalian cells are capable of modifying the fusion protein containing the BP peptide. The second bicistronic expression vector pFLAG-p53-BP-bir A encodes an identical dual-tagged p53 fusion protein (FIG. 2, right). In addition, it encodes E. coli Bir A (bir A) enzyme from the internal ribosome binding site. Both p53 and Bir A are made separately at high levels under transcriptional control of the same human cytomegalovirus (CMV promoter).

For construction of pFLAG-p53-BP, expression vector pFLAG-p53-c-Myc was constructed first by insertion of cDNA encoding wild-type full-length mouse p53 into pFLAG-Myc-CMV. Briefly, The cDNA encoding full-length mouse p53 was generated from mouse liver total RNA by RT-PCR method. Primer sequences for PCR are: p53 forward (5′-ATGCAAGCTTATGACTGCCATGGAGGAGTCACAGT-3′) (SEQ ID NO: 9) and p53 reverse (5′-CTAGTCTAGAGTCTGAGTCAGGCCCCACTTTCTTG-3′) (SEQ ID NO: 10) using a ReadyMix™ Taq PCR Reaction Mix. Restriction sites for Hind III and XbaI were incorporated in the forward and reverse primers, respectively. The resulting PCR fragment was then digested with Hind III and Xba I and ligated into Hind III and Xba I sites of pFLAG-Myc-CMV. A pair of complementary oligonucleotides containing the sequence encoding the 17 amino acid residues BP sequence was used to replace the c-Myc epitope of the resulting expression vector, pFLAG-p53-Myc-CMV. To this end, a pair of oligos was synthesized as follows: 5′-CTAGTCTAGAGGCAGTGGCCTGAATGATATCTTTGAAGCACAAAAAATTG AATGGCATGAATAACCCGGGGGAC-3′ (SEQ ID NO: 11) and 5′-GTCCCCCGGGTTATTCATGCCATTCAATTTTTTGTGCTTCAAAGATATCATT CAGGCCACTGCCTCTAGACTAG-3′(SEQ ID NO: 12). The oligos were annealed, digested with XbaI and Sma I and ligated to the XbaI/SmaI digested pFLAG-Myc-CMV to generate vector pFLAG-p53-BP-CMV. The final construct, bicistronic expression vector, pFLAG-p53-BP, was constructed by PCR amplification of FLAG-p53-BP sequence using the FLAG-p53-BP-CMV as the template. A pair of forward and reverse primers (forward: 5′-GATCGAATTCGAGCTCGTTTAGTGAACCGTCAGAA-3′; reverse primers: 5′-ACCGGAATTCTTATTCATGCCATTCAATTTTTTGTGCTTCAAA-3′) (SEQ ID NO: 13) were used for the PCR. Both primers carried an EcoR I restriction site. The resulting PCR fragment was then digested with EcoR I and inserted into the EcoRI digested pIRES vector (Clonetech). pFLAG-p53-BP-bir A was constructed by insertion of the E.coli bir A gene into pFLAG-p53-BP, downstream of the IRES sequence. A pair of oligonuclitides, bir A forward (5′-GATCACTCGTCGACAACCATGAAGGATAACACCGTGCCACTGAAATT-3′) (SEQ ID NO: 14) and bir A reverse (5′-GATCACTCGTCGACTTATTTTTCTGCACTACGCAGGGATATTT-3′) (SEQ ID NO: 15) were used as primers to isolate the bir A gene from E. coli genomic DNA using PCR. Restriction enzyme site Sal I was incorporated into both the forward and reverse primers. The resulting PCR fragment was then digested with Sal I and ligated into the Sal I site of pFLAG-p53-BP. For preparation of pc-Myc-CMV-2-large T-antigen, a N-terminal c-Myc tagged fusion protein containing the C-terminal of large T-antigen (residues: 86-708), we first constructed an N-terminal c-Myc vector, pc-Myc-CMV-2, by replacing the FLAG-sequence in pFLAG-CMV-2 with a c-Myc epitope (EQKLISEEDL) (23). To this end, pFLAG-CMV™-2 was digested with Sac I and Hind III to remove the FLAG sequence and then a pair of complimentary oligonucleotides containing the sequence encoding the ten amino acid c-Myc epitope was synthesized as follows: 5′-ACTGGAGCTCGTTTAGTGAACCGTCAGAATTAACCATGGAACAAAAACTC ATCTCAGAAGAGGA-3′TCTGAAGCTTACTG-3′(SEQ ID NO: 16) and 5′-CAGTAAGCTTCAGATCCTCTTCTGAGATGAGTTTTTGTTCCATGGTTAATT CTGACGGTTCACTAAACGAGCTCCAGT-3′(SEQ ID NO: 17). The oligos were annealed, digested with Sac I and Hind III and ligated to the Sac I/Hind III digested pFLAG-CMV-2 vector. Large T-antigen was PCR amplified from the plasmid pVP-16 T-AD vector (Clontech) and inserted into the Xba I site of pc-Myc-CMV-2. The sequences of all the constructed plasmids were confirmed by DNA sequencing using an ABI 373 DNA sequencer (Applied Biosystems, Foster City, Calif.).

### EXAMPLE 2

This example illustrates the culturing and transfection of host cells with the expression vectors.

COS-7 cells (American Type Culture Collection, Rockville, Md.) were maintained at 37° C. in DMEM supplemented with 10% fetal bovine serum, 100 units/ml penicillin, 100 μg/ml streptomycin, and 500 mg/liter L-glutamine. Cells were cultured in a humidified, 5% CO2 atmosphere tissue culture incubator and subcultured twice a week using trypsin-EDTA (0.05%, 0.53 mM) solution. The cells were plated in 6-well microplates at 4×10⁵/well 18 h before transfection with ESCORT II transfection reagent. 2 μg of plasmid were used with 10 μl of ESCORT II for each transfection experiment.

### EXAMPLE 3

This example illustrates the Western blot analysis of cell lysates prepared from cells transfected with expression vectors.

Cell lysates were prepared as follows. Growth medium was aspirated from the transfected cells to be assayed and the cells were rinsed twice with ice-cold PBS. 0.5 ml of ice-cold lysis buffer supplemented with mammalian protease inhibitors (10 μl per 1 ml lysis buffer) was added to each well of the 6-well plate. The lysis buffer contains 20 mM Tris-HCl, pH 7.4, 1 M NaCl, 1 mM DTT, 1.0% Triton X-100. Cells were scraped off the plate with cell scrapers, transferred to microcentrifuge tubes and incubated on ice for 1 h, followed by centrifugation at 14,000 rpm for 10 min at 4° C. A Bicinchoninic Acid Kit for Protein Determination was used to determine total protein concentration in the cell lysate.

Western blot analysis was carried out following standard procedures. Briefly, proteins were resolved on a 4-20% gradient SDS-PAGE and electrotransferred to PVDF membrane. The blot was blocked in blocking buffer (3% non-fat milk, 50 mM Tris-HCl, pH 7.5, 0.15 M NaCl) and probed with anti-FLAG M2-peroxidase or streptavidin-peroxidase conjugates diluted 1:2,000 in the blocking buffer. Reactive bands were detected with ECL plus™ according to the manufacturer's instruction (Amersham Pharmacia Biotech Inc., Piscataway, N.J., USA).

FIG. 2 shows Western blot analysis of the cell lysates prepared from the cells transfected with both expression vectors. Anti-FLAG M2 blot (FIG. 2 left) revealed FLAG-p53-BP fusion protein was expressed equivalently from both cells transfected with pFLAG-p53-BP and pFLAG-p53-BP-bir A (lane 2 and 3, respectively), whereas no protein band was detected with the anti-FLAG M2 HRP conjugate from the cell lysate prepared from the cells transfected with empty vector (lane 1). To determine whether any of the FLAG-p53-BP fusion protein was biotinylated, Western blot analysis of identical samples was performed using streptavidin-HRP conjugate (FIG. 2, right). As shown in FIG. 2 (lane 3), the biotinylation is highly specific, as the only expressed fusion protein being biotinylated is from the cells transfected with pFLAG-p53-BP-bir A. Even though BP peptide is well expressed as a portion of the fusion protein as revealed by the M2 blot (FIG. 2, lane 2), in the absence of enzyme Bir A, no biotinylation of the fusion protein was detected indicating the BP substrate is active only with Bir A. Furthermore, no detectable endogenous protein was biotinylated by the overexpressed Bir A enzyme indicating specificity of the Bir A enzyme for its substrate.

### EXAMPLE 4

This example illustrates capture of FLAG-p53-BP fusion protein on anti-FLAG M2 and Streptavidin (ST) plates.

One of the advantages of site-specific biotinylation in vivo is that it enables recombinant proteins to be immobilized in a direct orientation on streptavidin-coated surface and function analysis can be performed directly on the plate. To examine if the biotinylated FLAG-p53-BP fusion protein can be immobilized on a ST plate, the same cell lysate used for the Western blot analysis in FIG. 2 was used to incubate with both plates. Detection of the immobilized FLAG-p53-BP fusion was accomplished by incubating the plates with anti-FLAG M2 HRP conjugates. As for the controls, cell lysates prepared from the cells transfected with the empty vectors was also incubated with both plates.

Immobilization procedures were as follows. Both M2 and streptavidin plates were first washed one time with wash buffer containing 20 mM Tris-HCl (pH 7.4), 0.15 M NaCl, and 0.1% Triton X-100 to remove the preservatives on the plate. The plates were incubated with various amount of cell lysate prepared as above for 1 h at room temperature on a platform shaker. Nonspecific proteins were removed by washing the plate four times with wash buffer. 200 μl of M2-HRP or ST-HRP diluted 1:2000 in blocking buffer were added to the ST or M2 plates, respectively. At the end of the incubation, wells were washed four times with the washing buffer followed by a final wash with TBS buffer. Peroxidase substrate 3,3′,5,5′-Tetramethylbenzidine (TMB) was then added at 200 μl/well and allowed to incubate at room temperature. Reactions were stopped with 100 μl of H₂SO₄. The absorbance reading was taken at 450 nm (OD 450) with a Microplate Bio-kinetics Reader (Bio-Tek Instrument Inc., Winooski, Vt.). Blocking buffer contains 3% Nonfat Milk in TBST (Tris Buffered Saline, with Tween 20, pH 8.0).

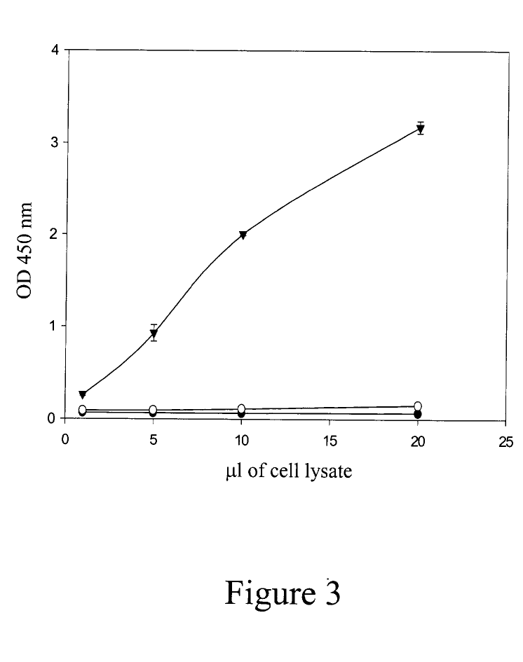

As shown in FIG. 3, ELISA analysis revealed that the only FLAG-p53-BP fusion protein specifically binds to the wells in ST plate are the ones expressed in the cells transfected with pFLAG-p53-BP-bir A. No appreciable signal was detected from the wells incubated with cell extracts prepared from cells transfected with FLAG-p53-BP, even though the fusion protein was well expressed as examined in the Western blot analysis (FIG. 2A). This result further confirmed the results in FIG. 2 in which biotinylation of FLAG-p53-BP is strictly dependent on the expression of Bir A from the bicistronic vector and the reaction can not be replaced by an endogenous Bir A-like enzyme of COS-7 cells. In addition, The binding of the FLAG-p53-BP fusion protein on the ST plate is quantitative, since the amount of fusion proteins captured is dependent on the amount of the cell lysates added to the plates. Moreover, biotinylation is quite efficient since signal can be detected in as little as 5 μl of the cell lysate

### EXAMPLE 5

This example illustrates the effect of increasing the concentration of biotin in the cell culture medium on the efficiency of biotinylation of the fusion protein in transfected cells.

The examples above showed that FLAG-p53-BP expressed in COS-7 cells was able to be biotinylated under conditions in which the cells were cultured in the DMEM media supplemented with 10% FBS which contains 0.15 μM of biotin. This example evaluated whether supplementing the media with higher concentration of biotin could increase the biotinylation efficiency of the fusion protein. To this end, 1 μM or 5 μM of biotin was added to the DMEM media supplemented with 10% FBS and used to culture the COS-7 cells transfected with pFLAG-p53-BP and pFLAG-p53-BP-bir A. 48 hrs post transfection, cell lysates were prepared and the level of the biotinylation of the FLAG-p53-BP fusion protein were determined on an anti-FLAG M2 plate with Streptavidin-HRP conjugate as described above.

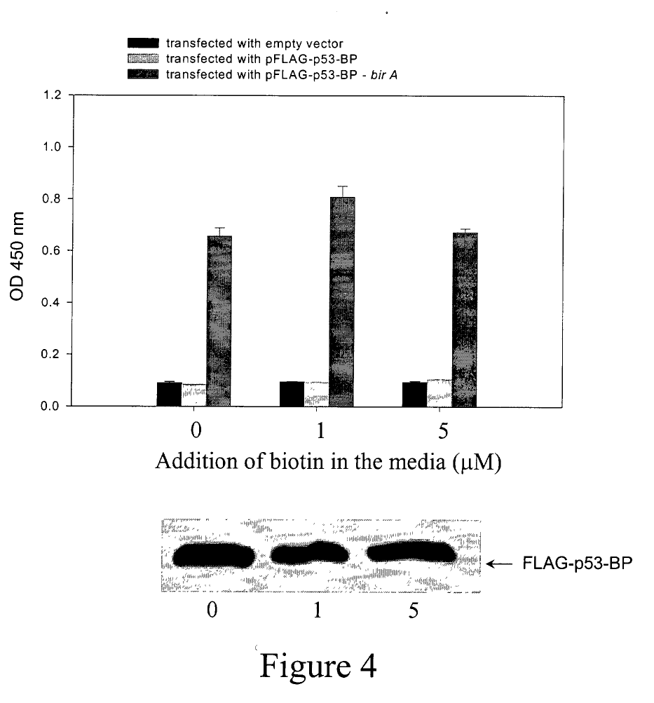

Consistent with the result in FIG. 3, when there was no exogenous biotin added, the only biotinylated FLAG-p53-BP fusion protein was from the cell lysates prepared from the cells transfected with the pFLAG-p53-BP-bir A (FIG. 4, upper panel). When 1 μM of biotin was added to the culture media, there was only slightly increased in the level of biotinylated FLAG-p53-BP. No further enhancement of biotinylated fusion protein was observed with higher amount of biotin (5 μM) (FIG. 4). Western blot analysis with anti-FLAG M2 antibody showed the protein expression level of the biotinylated FLAG-p53-BP fusion protein is comparable in each cell lysate prepared from the cells transfected with the pFLAG-p53-BP-bir A and cultured with different concentration of biotin (FIG. 4, lower panel). These data indicate no exogenous biotin is absolutely required for the in vivo biotinylation reaction since even a low level of biotin (0.15 μM) achieved closed to maximal level of biotinylation.

### EXAMPLE 6

This example illustrates the functional characterization of the interaction of FLAG-p53-BP fusion protein and large T antigen on both anti-FLAG M2 plate (M2 plate) and streptavidin plate.

The potentially deleterious effect of attaching a 17-mer biotinylation peptide to a protein is that additional amino acids and biotinylation may have on the functionality of the targeted protein. This was examined by testing the ability of the biotinylated FLAG-p53-BP fusion protein to interact with its in vivo interacting partner, the large T-antigen. To this end, an ELISA analysis was developed on both anti-FLAG M2 and streptavidin coated plates to study the interactions of the biotinylated FLAG-p53-BP fusion protein with both endogenous large T-antigen and exogenous c-Myc-tagged large T antigen.

To detect c-Myc-large T-antigen on the plates, 200 μl/well of monoclonal anti-c-Myc HRP conjugated diluted 1:1000 in blocking buffer were added. At the end of incubation, the plates were washed three times with wash buffer. After the plates were washed three times with wash buffer, 200 μl of anti-Myc-HRP antibody (1:1000 in blocking buffer) were added. At the end of incubation, the plates were washed 4 times with wash buffer followed by incubation with peroxidase substrate TMB at 200 μl/well for 10 min. OD readings was obtained as above.

To detect the interaction of FLAG-p53-BP and endogenous large T-antigen, biotin conjugated mouse anti-SV40 large T and small T antibody (clone Pab108, Research Diagnostics Inc., Flanders, N.J.) (1:10,000 in blocking buffer) was added at 200 μl/well and incubated for 1 h at room temperature. After the plates were washed three times with wash buffer, 200 μl of HRP-conjugated streptavidin (1:1000 in blocking buffer) were added. At the end of incubation, the plates were washed 4 times with wash buffer followed by incubation with peroxidase substrate TMB at 200 μl/well for 10 min. OD readings was obtained as above.

It is known that COS-7 cells, a SV40-transformed African Green monkey kidney fibroblasts, express the two major SV40 translational products including the 94 kDa large T-antigen and the 21 kDa small T-antigen, both of which are encoded by the early region of the SV40 viral genome (Gluzman Cell 23:175-82, 1981). The large T-antigen complex with the p53 suppressor gene resulting in its functional inactivation and thus promotion of cell transformation (Wang et al. Cell 57:379-392, 1989). In the ELISA assay, FLAG-p53-BP fusion proteins were captured directly from total cell lysates onto an anti-FLAG M2 plate. Bound endogenous large T-antigen to the captured FLAG-p53-BP fusion protein was detected directly by incubating the plate with biotinylated anti-SV40 T-antigen antibody followed by streptavidin-HRP conjugate.

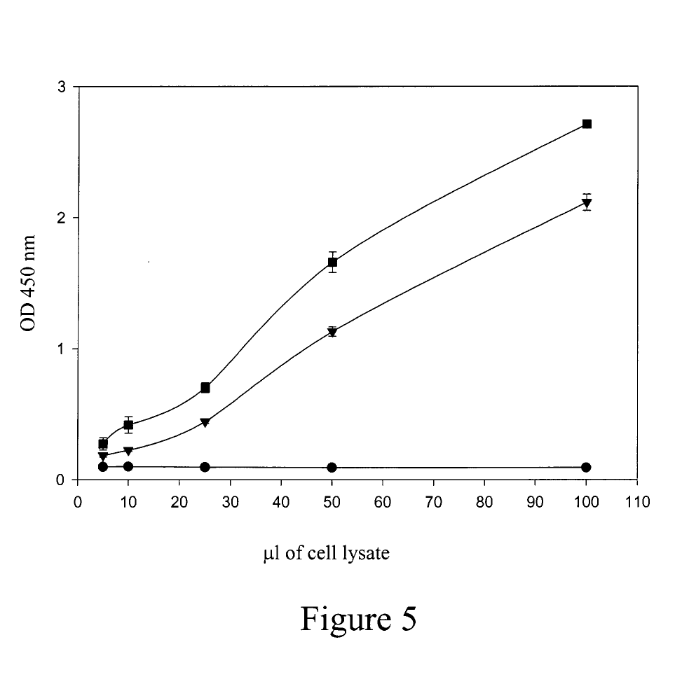

As shown in FIG. 5A, The expressed fusion proteins from cells transfected with pFLAG-p53-BP and pFLAG-p53-BP-bir A can be captured efficiently on the M2 plate. The amount of FLAG-p53-BP fusion proteins from both cell lysates prepared from the cells transfected with both plasmids precipitated onto the M2 plate is dependent upon the amount of lysate added to the plate, as well as the endogenous large T-antigen that co-precipiated with FLAG-p53-BP. When only empty vector was used to transfect the cells, no large T-antigen was immunoprecipitated with M2 plate. It is important to note that the small T-antigen, which is overexpressed in COS-7 cells and can be recognized by the anti-SV40 T-antigen antibody, was not coprecipitated with FLAG-p53-BP on the M2 plate indicating that interaction between FLAG-p53-BP and large T-antigen was specific.

The binding activity of the biotinylated FLAG-p53-BP to large T-antigen was then examined by designing an experiment in which the binding activity was determined on a ST plate in an ELISA assay. In this experiment, COS-7 cells were co-transfected with pFLAG-p53-BP-bir A and pc-Myc-large T antigen, the cell lysate was prepared and incubated with a ST plate. Detection of the bound c-Myc-large T-antigen to the captured biotinylated FLAG-p53-BP fusion protein was accomplished directly on the plate using anti-c-Myc HRP antibody conjugate in the ELISA analysis as described above.

As seen with the interaction of FLAG-p53-BP and endogenous large T-antigen in FIG. 5A, the ST plate also specifically detects the interaction of biotinylated FLAG-p53-BP and exogenous c-Myc-large T antigen (FIG. 5B). Like the interaction between FLAG-p53-BP and endogenous large T-antigen, bound c-Myc-large T antigen in the ST plate is linear with respect to total cell lysate incubated. Since the expressed FLAG-p53-BP fusion protein was not biotinylated in the cells transfected with pFLAG-p53-BP and c-Myc-large T-antigen, therefore no FLAG-p53-BP was captured and thus no detectable bound c-Myc-large T antigen was observed. Taken together, these results showed the binding properties of the biotinylated FLAG-p53-BP fusion protein to bind to large T-antigen was minimally affected by the immobilization on solid surfaces.

### EXAMPLE 7

This example illustrates fluorescent dual labeling of FLAG-p53-BP fusion protein expressed in COS-7 cells with anti-FLAG M2 FITC and ST-Cy3 conjugates.

To test whether biotinylated FLAG-p53-BP could be intracellularly detected by anti-FLAG M2 and ST-conjugates, COS-7 cells were transfected with pFLAG-p53-BP-bir A, subjected to dual fluorescent labeling with FITC-conjugated anti-FLAG M2 antibody and Cys-conjugated streptavidin simultaneously, and examined by fluorescent microscope

COS-7 cells seeded in an 8-chamber slide were transfected using ESCORT II as described above in Example 2. 24 or 48 h after transfection, cells were washed 3 times with phosphate-buffered saline (PBS), followed by fixation in 3% paraformadehyde in PBS for 30 min; The fixed cells were then washed 3 times with PBS and incubated with 0.1% Triton X-100 in PBS for 30 min at room temperature. The cells were then incubated with Streptavidin-Cy3 and anti-FLAG-M2-FITC diluted 1:100 in PBS for 1 h at room temperature. Cells were then viewed with a flurensence microscope under fluorescent light (excitation, 480 nm; emission, 510 nm for the FITC; or excitation 490 nm, and emission of 510 nm for the emission for the Cy3).

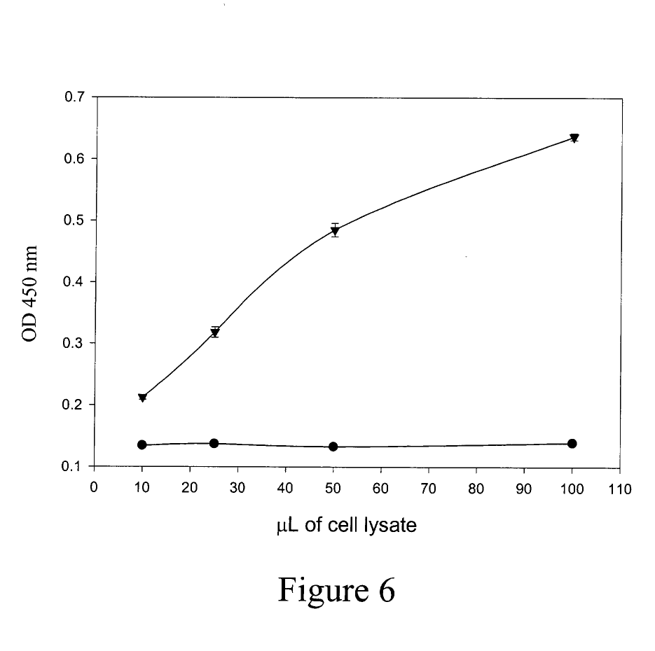

FIGS. 6A and B show the corresponding images to a single optical section of the representative cells for anti-FLAG M2 FITC and ST-Cy3 conjugates staining, respectively. The result revealed the exact identical staining pattern for both conjugates. This observation is further confirmed by superimposition of the two images as indicated by the yellow color (FIGS. 6C and D). Non transfected cells, no detectable staining was observed (data not shown). Thus, biotinylated FLAG-p53-BP fusion protein can be detected specifically in intact cell.

As various changes could be made in the above methods and compositions without departing from the scope of the invention, it is intended that all matter contained in the above description be interpreted as illustrative and not in a limiting sense.

All references cited in this specification are hereby incorporated by reference. The discussion of the references herein is intended merely to summarize the assertions made by their authors and no admission is made that any reference constitutes prior art relevant to patentability. Applicant reserves the right to challenge the accuracy and pertinency of the cited references.

What is claimed is:

1. A DNA molecule comprising a eukaryotic promoter operatively linked to a polynucleotide comprising:
a) a DNA sequence encoding a fusion protein comprising a selected protein and a biotinylation peptide;
b) a DNA sequence coding for an internal ribosome entry site and
c) a DNA sequence encoding a biotin ligase.

2. A DNA molecule of claim 1, wherein the fusion protein further comprises a protease cleavage site between the selected protein and the biotinylation peptide.

3. A DNA molecule of claim 1, wherein the fusion protein further comprises a leader peptide.

4. A DNA molecule of claim 1, wherein the biotinylation peptide comprises SEQ ID NO: 1 or a conservatively substituted variant thereof.

5. A DNA molecule of claim 1, wherein the internal ribosome entry site comprises a picornavirus internal ribosome entry site.

6. A DNA molecule of claim 5, wherein the picornavirus internal ribosome entry site comprises:
(SEQ ID NO: 4)
AATTCCGCCCCTCTCCCTCCCCCCCCCCTAACGTTACTGGCCGAAGCCGC
TTGGAATAAGGCCGGTGTGCGTTTGTCTATATGTGATTTTCCACCATATT
GCCGTCTTTTGGCAATGTGAGGGCCCGGAAACCTGGCCCTGTCTTCTTGA
CGAGCATTCCTAGGGGTCTTTCCCCTCTCGCCAAAGGAATGCAAGGTCTG
TTGAATGTCGTGAAGGAAGCAGTTCCTCTGGAAGCTTCTTGAAGACAAAC
AACGTCTGTAGCGACCCTTTGCAGGCAGCGGAACCCCCCACCTGGCGACA
GGTGCCTCTGCGGCCAAAAGCCACGTGTATAAGATACACCTGCAAAGGCG
GCACAACCCCAGTGCCACGTTGTGAGTTGGATAGTTGTGGAAAGAGTCAA
ATGGCTCTCCTCAAGCGTATTCAACAAGGGGCTGAAGGATGCCCAGAAGG
TACCCCATTGTATGGGATCTGATCTGGGGCCTCGGTGCACATGCTTTACA
TGTGTTTAGTCGAGGTTAAAAAAACGTCTAGGCCCCCCGAACCACGGGGA
CGTGGTTTTCCTTTGAAAAACACGATGATAA.

7. A DNA molecule of claim 1, wherein the biotin ligase comprises a bacterial biotin ligase.

8. A DNA molecule of claim 7, wherein the biotin ligase comprises a Bir A.

9. A DNA molecule of claim 1, wherein the eukaryotic promoter comprises a cytomegalovirus immediate early promoter.

10. A DNA molecule of claim 1, which is a double stranded DNA molecule.

11. A DNA molecule of claim 1, wherein the fusion protein further comprises an epitope tag.

12. A DNA molecule of claim 11, wherein the epitope tag is a FLAG sequence, a myc epitope tag, or a polyhistidine tag.

13. A vector comprising the DNA molecule of claim 1.

14. A host cell comprising the DNA molecule of claim 1.

15. A host cell of claim 14 which is a mammalian cell.

16. A host cell of claim 15 wherein the mammalian cell is a COS cell.

17. A DNA molecule for insertion of a DNA sequence encoding a selected protein, the DNA molecule comprising a eukaryotic promoter operatively linked to a polynucleotide comprising:
a) a site allowing for insertion of the DNA sequence encoding a selected protein;
b) a DNA sequence encoding a biotinylation peptide;
c) a DNA sequence coding for an internal ribosome entry site; and
d) a DNA sequence encoding a biotin ligase,
wherein upon insertion of the DNA sequence encoding a selected protein, the DNA encoding the selected protein and the DNA sequence encoding the biotinylation peptide comprise a DNA encoding a fusion protein.

18. A DNA molecule of claim 17, wherein the fusion protein further comprises a protease cleavage site between the selected protein and the biotinylation peptide.

19. A DNA molecule of claim 17, wherein the fusion protein further comprises a leader peptide.

20. A DNA molecule of claim 1, wherein the biotinylation peptide comprises SEQ ID NO: 1 or a conservatively substituted variant thereof.

21. A DNA molecule of claim 17, wherein the internal ribosome entry site comprises a picornavirus internal ribosome entry site.

22. A DNA molecule of claim 21, wherein the picornavirus internal ribosome entry site comprises:
(SEQ ID NO: 4)
AATTCCGCCCCTCTCCCTCCCCCCCCCCTAACGTTACTGGCCGAAGCCGC
TTGGAATAAGGCCGGTGTGCGTTTGTCTATATGTGATTTTCCACCATATT
GCCGTCTTTTGGCAATGTGAGGGCCCGGAAACCTGGCCCTGTCTTCTTGA
CGAGCATTCCTAGGGGTCTTTCCCCTCTCGCCAAAGGAATGCAAGGTCTG
TTGAATGTCGTGAAGGAAGCAGTTCCTCTGGAAGCTTCTTGAAGACAAAC
AACGTCTGTAGCGACCCTTTGCAGGCAGCGGAACCCCCCACCTGGCGACA
GGTGCCTCTGCGGCCAAAAGCCACGTGTATAAGATACACCTGCAAAGGCG
GCACAACCCCAGTGCCACGTTGTGAGTTGGATAGTTGTGGAAAGAGTCAA
ATGGCTCTCCTCAAGCGTATTCAACAAGGGGCTGAAGGATGCCCAGAACG
TACCCCATTGTATGGGATCTGATCTGGGGCCTCGGTGCACATGCTTTACA
TGTGTTTAGTCGAGGTTAAAAAAACGTCTAGGCCCCCCGAACCACGGGGA
CGTGGTTTTCCTTTGAAAAACACGATGATAA.

23. A DNA molecule of claim 17, wherein the biotin ligase comprises a bacterial biotin ligase.

24. A DNA molecule of claim 23, wherein the biotin ligase comprises a Bir A.

25. A DNA molecule of claim 17, wherein the eukaryotic promoter comprises a cytomegalovirus immediate early promoter.

26. A DNA molecule of claim 17, wherein the fusion protein further comprises an epitope tag.

27. A DNA molecule of claim 26, wherein the epitope tag is a FLAG sequence, a myc epitope tag, or a polyhistidine tag.

28. A DNA molecule of claim 17, wherein the site allowing for insertion of the DNA sequence is a restriction enzyme cleavage site.

29. A DNA molecule of claim 17, wherein the site allowing for insertion of the DNA sequence is a recombination site.

30. A DNA molecule of claim 17 wherein the site allowing for insertion of the DNA is a topoisomerase I-based site.

31. A DNA molecule of claim 17, which is double stranded DNA.

32. A DNA molecule of claim 17 which is a circular DNA molecule.

33. A DNA molecule of claim 17 which is a linear DNA molecule.

34. A vector comprising the DNA molecule of claim 17.

35. A host cell comprising the DNA molecule of claim 17.

36. A host cell of claim 35 which is a mammalian cell.

37. A host cell of claim 36 wherein the mammalian cell is a COS cell.

38. A DNA cassette for generating a DNA molecule encoding a fusion protein comprising a selected protein and a biotinylation peptide, the cassette comprising:
a) a DNA sequence encoding a biotinylation peptide;
b) a DNA sequence coding for an internal ribosome entry site; and
c) a DNA sequence encoding a biotin ligase.

39. A DNA cassette of claim 38, wherein the fusion protein further comprises a protease cleavage site between the selected protein and the biotinylation peptide.

40. A DNA cassette of claim 38, wherein the fusion protein further comprises a leader peptide.

41. A DNA cassette of claim 1, wherein the biotinylation peptide comprises SEQ ID NO: 1 or a conservatively substituted variant thereof.

42. A DNA cassette of claim 38, wherein the internal ribosome entry site comprises a picornavirus internal ribosome entry site.

43. A DNA cassette of claim 42, wherein the picornavirus internal ribosome entry site comprises:
(SEQ ID NO: 4)
AATTCCGCCCCTCTCCCTCCCCCCCCCCTAACGTTACTGGCCGAAGCCGC
TTGGAATAAGGCCGGTGTGCGTTTGTCTATATGTGATTTTCCACCATATT
GCCGTCTTTTGGCAATGTGAGGGCCCGGAAACCTGGCCCTGTCTTCTTGA
CGAGCATTCCTAGGGGTCTTTCCCCTCTCGCCAAAGGAATGCAAGGTCTG
TTGAATGTCGTGAAGGAAGCAGTTCCTCTGGAAGCTTCTTGAAGACAAAC
AACGTCTGTAGCGACCCTTTGCAGGCAGCGGAACCCCCCACCTGGCGACA
GGTGCCTCTGCGGCCAAAAGCCACGTGTATAAGATACACCTGCAAAGGCG
GCACAACCCCAGTGCCACGTTGTGAGTTGGATAGTTGTGGAAAGAGTCAA
ATGGCTCTCCTCAAGCGTATTCAACAAGGGGCTGAAGGATGCCCAGAAGG
TACCCCATTGTATGGGATCTGATCTGGGGCCTCGGTGCACATGCTTTACA
TGTGTTTAGTCGAGGTTAAAAAAACGTCTAGGCCCCCCGAACCACGGGGA
CGTGGTTTTCCTTTGAAAAACACGATGATAA.

44. A DNA cassette of claim 38, wherein the biotin ligase comprises a bacterial biotin ligase.

45. A DNA cassette of claim 44, wherein the biotin ligase comprises a Bir A.

46. A DNA cassette of claim 38, wherein the eukaryotic promoter comprises a cytomegalovirus immediate early promoter.

47. A DNA cassette of claim 38, wherein the fusion protein further comprises an epitope tag.

48. A DNA cassette of claim 47, wherein the epitope tag is a FLAG sequence, a myc epitope tag, or a polyhistidine tag.

49. A DNA cassette of claim 38 which is double stranded DNA.

50. A DNA cassette of claim 38 which is an amplicon.

51. A DNA cassette of claim 38 which is a linear DNA molecule.

52. A vector comprising the DNA cassette of claim 38.

53. A host cell comprising the DNA cassette of claim 38.

54. A host cell of claim 53 which is a mammalian cell.

55. A host cell of claim 54 wherein the mammalian cell is a COS cell.

56. An RNA molecule comprising:
a) an RNA sequence encoding a fusion protein comprising a selected protein and a biotinylation peptide; and
b) an RNA sequence encoding a biotin ligase operatively linked to an internal ribosome entry site.

57. An RNA molecule of claim 56, wherein the fusion protein further comprises a protease cleavage site between the selected protein and the biotinylation peptide.

58. An RNA molecule of claim 56, wherein the fusion protein further comprises a leader peptide.

59. An RNA molecule of claim 56, wherein the biotinylation peptide comprises SEQ ID NO: 1 or a conservatively substituted variant thereof.

60. An RNA molecule of claim 56, wherein the internal ribosome entry site comprises a picornavirus internal ribosome entry site.

61. An RNA molecule of claim 60, wherein the picornavirus internal ribosome entry site comprises
(SEQ ID NO: 5)
AAUUCCGCCCCUCUCCCUCCCCCCCCCCUAACGUUACUGGCCGAAGCCGC
UUGGAAUAAGGCCGGUGUGCGUUUGUCUAUAUGUGAUUUUCCACCAUAUU
GCCGUCUUUUGGCAAUGUGAGGGCCCGGAAACCUGGCCCUGUCUUCUUGA
CGAGCAUUCCUAGGGGUCUUUCCCCUCUCGCCAAAGGAAUGCAAGGUCUG
UUGAAUGUCGUGAAGGAAGCAGUUCCUCUGGAAGCUUCUUGAAGACAAAC
AACGUCUGUAGCGACCCUUUGCAGGCAGCGGAACCCCCCACCUGGCGACA
GGUGCCUCUGCGGCCAAAAGCCACGUGUAUAAGAUACACCUGCAAAGGCG
GCACAACCCCAGUGCCACGUUGUGAGUUGGAUAGUUGUGGAAAGAGUCAA
AUGGCUCUCCUCAAGCGUAUUCAACAAGGGGCUGAAGGAUGCCCAGAAGG
UACCCCAUUGUAUGGGAUCUGAUCUGGGGCCUCGGUGCACAUGCUUUACA
UGUGUUUAGUCGAGGUUAAAAAAACGUCUAGGCCCCCCGAACCACGGGGA
CGUGGUUUUCCUUUGAAAAACACGAUGAUAA.

62. An RNA molecule of claim 56, wherein the biotin ligase comprises a bacterial biotin ligase.

63. An RNA molecule of claim 62, wherein the biotin ligase comprises a Bir A.

64. An RNA molecule of claim 56, wherein the fusion protein further comprises an epitope tag.

65. An RNA molecule of claim 64, wherein the epitope tag is a FLAG sequence, a myc epitope tag, or a polyhistidine tag.

66. A host cell comprising the RNA molecule of claim 56.

67. A host cell of claim 66 which is a mammalian cell.

68. A host cell of claim 67 which is a COS cell.

69. A method of preparing a biotinylated fusion protein comprising:
a) providing a nucleic acid comprising a eukaryotic promoter operatively linked to a polynucleotide, the polynucleotide comprising:
i) a DNA sequence encoding a fusion protein comprising a selected protein and a biotinylation peptide;
ii) a DNA sequence coding for an internal ribosome entry site and
iii) a DNA sequence encoding a biotin ligase; and
b) incubating the nucleic acid under conditions which produce a biotinylated fusion protein in an incubation mixture.

70. A method of claim 69, wherein the biotinylated fusion protein further comprises a protease cleavage site between the selected protein and the biotinylation peptide.

71. A method of claim 70, further comprising proteolytically cleaving the fusion protein at the protease cleavage site.

72. A method of claim 69, wherein the fusion protein further comprises a leader peptide.

73. A method of claim 69, wherein the biotinylation peptide comprises SEQ ID NO: 1 or a conservatively substituted variant thereof.

74. A method of claim 69, wherein the internal ribosome entry site comprises a picornavirus internal ribosome entry site.

75. A method of claim 74, wherein the picornavirus internal ribosome entry site comprises:
(SEQ ID NO: 4)
AATTCCGCCCCTCTCCCTCCCCCCCCCCTAACGTTACTGGCCGAAGCCGC
TTGGAATAAGGCCGGTGTGCGTTTGTCTATATGTGATTTTCCACCATATT
GCCGTCTTTTGGCAATGTGAGGGCCCGGAAACCTGGCCCTGTCTTCTTGA
CGAGCATTCCTAGGGGTCTTTCCCCTCTCGCCAAAGGAATGCAAGGTCTG
TTGAATGTCGTGAAGGAAGCAGTTCCTCTGGAAGCTTCTTGAAGACAAAC
AACGTCTGTAGCGACCCTTTGCAGGCAGCGGAACCCCCCACCTGGCGACA
GGTGCCTCTGCGGCCAAAAGCCACGTGTATAAGATACACCTGCAAAGGCG
GCACAACCCCAGTGCCACGTTGTGAGTTGGATAGTTGTGGAAAGAGTCAA
ATGGCTCTCCTCAAGCGTATTCAACAAGGGGCTGAAGGATGCCCAGAAGG
TACCCCATTGTATGGGATCTGATCTGGGGCCTCGGTGCACATGCTTTACA
TGTGTTTAGTCGAGGTTAAAAAAACGTCTAGGCCCCCCGAACCACGGGGA
CGTGGTTTTCCTTTGAAAAACACGATGATAA.

76. A method of claim 69, wherein the biotin ligase comprises a bacterial biotin ligase.

77. A method of claim 76, wherein the biotin ligase comprises a Bir A.

78. A method of claim 69, wherein the eukaryotic promoter comprises a cytomegalovirus immediate early promoter.

79. A method of claim 69, wherein the nucleic acid is a double stranded DNA molecule.

80. A method of claim 69, wherein the fusion protein further comprises an epitope tag.

81. A method of claim 80, wherein the epitope tag is a FLAG sequence, a myc epitope tag, or a polyhistidine tag.

82. A method of claim 69, wherein the DNA molecule comprises a vector.

83. A method of claim 69, wherein the DNA molecule comprises a host cell.

84. A method of claim 82, wherein the host cell is a mammalian cell.

85. A method of claim 84, wherein the host cell is a COS cell.

86. A method of claim 69, wherein the nucleic acid comprises an in vitro expression system.

87. A method of claim 86, wherein the in vitro expression system is a reticulocyte lysate expression system, a wheat germ extract expression system, or a Xenopus oocyte extract expression system.

88. A method of claim 69, further comprising:
c) contacting the biotinylated fusion protein with a binding partner under conditions which permit binding; and
d) separating the biotinylated fusion protein bound to the binding partner from the incubation mixture.

89. A method of claim 88, wherein the binding partner is a biotin binding partner which is immobilized on a solid support.

90. A method of claim 88 wherein the biotinylated fusion protein further comprises an epitope tag, the binding partner is a binding partner to the epitope tag, and the binding partner to the epitope tag is immobilized on a solid support.

91. A method of claim 88, further comprising releasing the biotinylated fusion protein from the binding partner.

92. A method of claim 69, further comprising:
c) contacting the biotinylated fusion protein with a binding partner under conditions which permit binding; and
d) detecting the biotinylated fusion protein bound to the binding partner from the incubation mixture.

93. A method of claim 92, wherein the binding partner is a biotin binding partner.

94. A method of claim 92, wherein the biotinylated fusion protein further comprises an epitope tag and the binding partner is a binding partner to the epitope tag.

95. A method of producing a DNA molecule encoding a fusion protein comprising a selected protein and a biotinylated peptide, the method comprising:
a) providing a first DNA molecule comprising:
i) a DNA sequence comprising a eukaryotic promoter operatively linked to a site allowing for insertion of the DNA sequence encoding the selected protein and a DNA sequence encoding a biotinylation peptide;
ii) a DNA sequence coding for an internal ribosome entry site; and
iii) a DNA sequence encoding a biotin ligase;
b) providing the second DNA molecule comprising a DNA sequence encoding the selected protein;
c) linking the first DNA molecule and the second DNA molecule such that the DNA sequence encoding a selected protein and the DNA sequence encoding the biotinylation peptide comprise a DNA sequence encoding the fusion protein.

96. A method of claim 95, wherein the fusion protein further comprises a protease cleavage site between the selected protein and the biotinylation peptide.

97. A method of claim 95, wherein the fusion protein further comprises a leader peptide.

98. A method of claim 95, wherein the biotinylation peptide comprises SEQ ID NO: 1 or a conservatively substituted variant thereof.

99. A method of claim 95, wherein the internal ribosome entry site comprises a picornavirus internal ribosome entry site.

100. A method of claim 99, wherein the picornavirus internal ribosome entry site comprises:
(SEQ ID NO: 4)
AATTCCGCCCCTCTCCCTCCCCCCCCCCTAACGTTACTGGCCGAAGCCGC
TTGGAATAAGGCCGGTGTGCGTTTGTCTATATGTGATTTTCCACCATATT
GCCGTCTTTTGGCAATGTGAGGGCCCGGAAACCTGGCCCTGTCTTCTTGA
CGAGCATTCCTAGGGGTCTTTCCCCTCTCGCCAAAGGAATGCAAGGTCTG
TTGAATGTCGTGAAGGAAGCAGTTCCTCTGGAAGCTTCTTGAAGACAAAC
AACGTCTGTAGCGACCCTTTGCAGGCAGCGGAACCCCCCACCTGGCGACA
GGTGCCTCTGCGGCCAAAAGCCACGTGTATAAGATACACCTGCAAAGGCG
GCACAACCCCAGTGCCACGTTGTGAGTTGGATAGTTGTGGAAAGAGTCAA
ATGGCTCTCCTCAAGCGTATTCAACAAGGGGCTGAAGGATGCCCAGAAGG
TACCCCATTGTATGGGATCTGATCTGGGGCCTCGGTGCACATGCTTTACA
TGTGTTTAGTCGAGGTTAAAAAAACGTCTAGGCCCCCCGAACCACGGGGA
CGTGGTTTTCCTTTGAAAAACACGATGATAA.

101. A method of claim 95, wherein the biotin ligase comprises a bacterial biotin ligase.

102. A method of claim 101, wherein the biotin ligase comprises a Bir A.

103. A method of claim 95, wherein the eukaryotic promoter comprises a cytomegalovirus immediate early promoter.

104. A method of claim 95, wherein the fusion protein further comprises an epitope tag.

105. A method of claim 104, wherein the epitope tag is a FLAG sequence, a myc epitope tag, or a polyhistidine tag.

106. A method of claim 95, wherein the site allowing for insertion of the DNA sequence is a restriction enzyme cleavage site.

107. A method of claim 95, wherein the site allowing for insertion of the DNA sequence is a recombination site.

108. A method of claim 95 wherein the site allows for insertion of the DNA is a topoisomerase I-based site.

109. A method of claim 95, wherein the first DNA molecule is double stranded DNA.

110. A method of claim 95, wherein the first DNA molecule is a circular DNA molecule.

111. A method of claim 95, wherein the first DNA molecule is a linear DNA molecule.

112. A method of claim 95, wherein the first DNA molecule comprises a vector.

113. A method of producing a DNA molecule encoding a fusion protein comprising a selected protein and a biotinylation peptide, the method comprising:
a) providing a DNA cassette comprising:
i) a DNA sequence encoding a biotinylation peptide;
ii) a DNA sequence coding for an internal ribosome entry site; and
iii) a DNA sequence encoding a biotin ligase;
b) providing a second DNA molecule comprising a eukaryotic promoter operatively linked to DNA sequence encoding the selected protein;
c) inserting the DNA cassette into the DNA molecule encoding a selected protein such that the sequence encoding a biotinylation peptide and the sequence encoding a selected protein comprise a DNA encoding a fusion protein.

114. A method of claim 113, wherein the fusion protein further comprises a protease cleavage site between the selected protein and the biotinylation peptide.

115. A method of claim 113, wherein the fusion protein further comprises a leader peptide.

116. A method of claim 1, wherein the biotinylation peptide comprises SEQ ID NO: 1 or a conservatively substituted variant thereof.

117. A method of claim 113, wherein the internal ribosome entry site comprises a picornavirus internal ribosome entry site.

118. A method of claim 117, wherein the picornavirus internal ribosome entry site comprises:
(SEQ ID NO: 4)
AATTCCGCCCCTCTCCCTCCCCCCCCCCTAACGTTACTGGCCGAAGCCGC
TTGGAATAAGGCCGGTGTGCGTTTGTCTATATGTGATTTTCCACCATATT
GCCGTCTTTTGGCAATGTGAGGGCCCGGAAACCTGGCCCTGTCTTCTTGA
CGAGCATTCCTAGGGGTCTTTCCCCTCTCGCCAAAGGAATGCAAGGTCTG
TTGAATGTCGTGAAGGAAGCAGTTCCTCTGGAAGCTTCTTGAAGACAAAC
AACGTCTGTAGCGACCCTTTGCAGGCAGCGGAACCCCCCACCTGGCGACA
GGTGCCTCTGCGGCCAAAAGCCACGTGTATAAGATACACCTGCAAAGGCG
GCACAACCCCAGTGCCACGTTGTGAGTTGGATAGTTGTGGAAAGAGTCAA
ATGGCTCTCCTCAAGCGTATTCAACAAGGGGCTGAAGGATGCCCAGAAGG
TACCCCATTGTATGGGATCTGATCTGGGGCCTCGGTGCACATGCTTTACA
TGTGTTTAGTCGAGGTTAAAAAAACGTCTAGGCCCCCCGAACCACGGGGA
CGTGGTTTTCCTTTGAAAAACACGATGATAA.

119. A method of claim 113, wherein the biotin ligase comprises a bacterial biotin ligase.

120. A method of claim 119, wherein the biotin ligase comprises a Bir A.

121. A method of claim 113, wherein the eukaryotic promoter comprises a cytomegalovirus immediate early promoter.

122. A method of claim 113, wherein the fusion protein further comprises an epitope tag.

123. A method of claim 122, wherein the epitope tag is a FLAG sequence, a myc epitope tag, or a polyhistidine tag.

124. A method of claim 113, wherein the DNA cassette is double stranded DNA.

125. A method of claim 113, wherein the DNA cassette is an amplicon.

126. A method of claim 113, wherein the DNA cassette is a circular DNA molecule.

127. A method of claim 113, wherein the DNA cassette is a linear DNA molecule.

128. A method of claim 113, wherein the DNA cassette comprises a vector.

129. A method for producing a biotinylated protein comprising:
1) providing an RNA molecule comprising
a) an RNA sequence encoding a fusion protein comprising a selected protein and a biotinylation peptide; and
b) an RNA sequence encoding a biotin ligase operatively linked to an internal ribosome entry site and
2) incubating the RNA molecule under conditions which produce a biotinylated fusion protein in an incubation mixture.

130. A method of claim 129, wherein the fusion protein further comprises a protease cleavage site between the selected protein and the biotinylation peptide.

131. A method of claim 129, wherein the fusion protein further comprises a leader peptide.

132. A method of claim 129, wherein the biotinylation peptide comprises SEQ ID NO: 1 or a conservatively substituted variant thereof.

133. A method of claim 129, wherein the internal ribosome entry site comprises a picornavirus internal ribosome entry site.

134. A method of claim 133, wherein the picornavirus internal ribosome entry site comprises
(SEQ ID NO: 5)
AAUUCCGCCCCUCUCCCUCCCCCCCCCCUAACGUUACUGGCCGAAGCCGC
UUGGAAUAAGGCCGGUGUGCGUUUGUCUAUAUGUGAUUUUCCACCAUAUU
GCCGUCUUUUGGCAAUGUGAGGGCCCGGAAACCUGGCCCUGUCUUCUUGA
CGAGCAUUCCUAGGGGUCUUUCCCCUCUCGCCAAAGGAAUGCAAGGUCUG
UUGAAUGUCGUGAAGGAAGCAGUUCCUCUGGAAGCUUCUUGAAACAAACA
ACGUCUGUAGCGACCCUUUGCAGGCAGCGGAACCCCCCACCUGGCGACAG
GUGCCUCUGCGGCCAAAAGCCACGUGUAUAAGAUACACCUGCAAAGGCGG
CACAACCCCAGUGCCACGUUGUGAGUUGGAUAGUUGUGGAAAGAGUCAAA
UGGCUCUCCUCAAGCGUAUUCAACAAGGGGCUGAAGGAUGCCCAGAAGGU
ACCCCAUUGUAUGGGAUCUGAUCUGGGGCCUCGGUGCACAUGCUUUACAU
GUGUUUAGUCGAGGUUAAAAAAACGUCUAGGCCCCCCGAACCACGGGGAC
GUGGUUUUCCUUUGAAAAACACGAUGAUAA.

135. A method of claim 129, wherein the biotin ligase comprises a bacterial biotin ligase.

136. A method of claim 135, wherein the biotin ligase comprises a Bir A.

137. A method of claim 129, wherein the fusion protein further comprises an epitope tag.

138. A method of claim 137, wherein the epitope tag is a FLAG sequence, a myc epitope tag, or a polyhistidine tag.

139. A method of claim 129, wherein a host cell comprises the RNA molecule.

140. A method of claim 139, wherein the host cell which is a mammalian cell.

141. A method of claim 140, wherein the host cell is a COS cell.

142. A method of claim 129, wherein the RNA molecule comprises an in vitro translation system.

143. A method of claim 142, wherein the in vitro translation system is a reticulocyte lysate translation system, a wheat germ extract translation system, or a Xenopus oocyte extract translation system.

144. A kit for producing a biotinylated fusion protein, the kit comprising a DNA molecule comprising a promoter operatively linked to a polynucleotide, the polynucleotide comprising:
a) a DNA sequence encoding a fusion protein comprising a selected protein and a biotinylation peptide;
b) a DNA sequence coding for an internal ribosome entry site;
c) a DNA sequence encoding a biotin ligase; and
wherein the DNA molecule is packaged in a container.

145. A kit of claim 144, wherein the biotinylated fusion protein further comprises a protease cleavage site between the selected protein and the biotinylation peptide.

146. A kit of claim 144, further comprising a protease enzyme.

147. A kit of claim 144, wherein the biotinylated fusion protein further comprises a leader peptide.

148. A kit of claim 144, wherein the biotinylation peptide comprises SEQ ID NO: 1 or a conservatively substituted variant thereof.

149. A kit of claim 144, wherein the internal ribosome entry site comprises a picornavirus internal ribosome entry site.

150. A kit of claim 149, wherein the picornavirus internal ribosome entry site comprises:
(SEQ ID NO: 4)
AATTCCGCCCCTCTCCCTCCCCCCCCCCTAACGTTACTGGCCGAAGCCGC
TTGGAATAAGGCCGGTGTGCGTTTGTCTATATGTGATTTTCCACCATATT
GCCGTCTTTTGGCAATGTGAGGGCCCGGAAACCTGGCCCTGTCTTCTTGA
CGAGCATTCCTAGGGGTCTTTCCCCTCTCGCCAAAGGAATGCAAGGTCTG
TTGAATGTCGTGAAGGAAGCAGTTCCTCTGGAAGCTTCTTGAAGACAAAC
AACGTCTGTAGCGACCCTTTGCAGGCAGCGGAACCCCCCACCTGGCGACA
GGTGCCTCTGCGGCCAAAAGCCACGTGTATAAGATACACCTGCAAAGGCG
GCACAACCCCAGTGCCACGTTGTGAGTTGGATAGTTGTGGAAAGAGTCAA
ATGGCTCTCCTCAAGCGTATTCAACAAGGGGCTGAAGGATGCCCAGAAGG
TACCCCATTGTATGGGATCTGATCTGGGGCCTCGGTGCACATGCTTTACA
TGTGTTTAGTCGAGGTTAAAAAAACGTCTAGGCCCCCCGAACCACGGGGA
CGTGGTTTTCCTTTGAAAAACACGATGATAA.

151. A kit of claim 144, wherein the biotin ligase comprises a bacterial biotin ligase.

152. A kit of claim 151, wherein the biotin ligase comprises a Bir A.

153. A kit of claim 144, wherein the eukaryotic promoter comprises a cytomegalovirus immediate early promoter.

154. A kit of claim 144, wherein the DNA molecule is double stranded DNA.

155. A kit of claim 144, wherein the DNA molecule comprises a vector.

156. A kit of claim 144, further comprising a host cell comprising the DNA molecule.

157. A kit of claim 156, wherein the host cell is a mammalian cell.

158. A kit of claim 157, wherein the mammalian cell is a COS cell.

159. A kit of claim 144, further comprising an in vitro expression system.

160. A kit of claim 144, wherein the in vitro expression system is a reticulocyte lysate expression system, a wheat germ extract expression system, or a Xenopus oocyte extract expression system.

161. A kit of claim 144, further comprising a biotin binding partner for the biotinylated fusion protein.

162. A kit of claim 161, wherein the biotin binding partner is immobilized on a solid support.

163. A kit of claim 161, wherein the biotinylated fusion protein further comprises an epitope tag.

164. A kit of claim 163, wherein the epitope tag is a FLAG sequence, a myc epitope tag, or a polyhistidine tag.

165. A kit of claim 163, further comprising a binding partner to the epitope tag.

166. A kit of claim 165, wherein the biotin binding partner is immobilized on a solid support and the binding partner to the epitope tag is immobilized on a solid support.

167. A kit for producing a nucleic acid molecule for generating a fusion protein comprising a selected protein and a biotinylated peptide, the kit comprising:
a DNA molecule comprising a eukaryotic promoter 5′ to a polynucleotide comprising:
a) a DNA sequence comprising a site allowing for insertion of a DNA sequence encoding a selected protein and a DNA sequence encoding a biotinylation peptide, wherein upon insertion, the DNA encoding the selected protein and the DNA sequence encoding the biotinylation peptide are in the same reading frame and encode a fusion protein;
b) a DNA sequence coding for an internal ribosome entry site; and
c) a DNA sequence encoding a biotin ligase;
wherein the DNA molecule is packaged in a container.

168. A kit of claim 167, wherein the fusion protein further comprises a protease cleavage site between the selected protein and the biotinylation peptide.

169. A kit of claim 167, wherein the fusion protein further comprises a leader peptide.

170. A kit of claim 167, wherein the biotinylation peptide comprises SEQ ID NO: 1 or conservatively substituted variants thereof.

171. A kit of claim 167, wherein the internal ribosome entry site comprises a picornavirus internal ribosome entry site.

172. A kit of claim 171, wherein the picornavirus internal ribosome entry site comprises:
(SEQ ID NO: 4)
AATTCCGCCCCTCTCCCTCCCCCCCCCCTAACGTTACTGGCCGAAGCCGC
TTGGAATAAGGCCGGTGTGCGTTTGTCTATATGTGATTTTCCACCATATT
GCCGTCTTTTGGCAATGTGAGGGCCCGGAAACCTGGCCCTGTCTTCTTGA
CGAGCATTCCTAGGGGTCTTTCCCCTCTCGCCAAAGGAATGCAAGGTCTG
TTGAATGTCGTGAAGGAAGCAGTTCCTCTGGAAGCTTCTTGAAGACAAAC
AACGTCTGTAGCGACCCTTTGCAGGCAGCGGAACCCCCCACCTGGCGACA
GGTGCCTCTGCGGCCAAAAGCCACGTGTATAAGATACACCTGCAAAGGCG
GCACAACCCCAGTGCCACGTTGTGAGTTGGATAGTTGTGGAAAGAGTCAA
ATGGCTCTCCTCAAGCGTATTCAACAAGGGGCTGAAGGATGCCCAGAAGG
TACCCCATTGTATGGGATCTGATCTGGGGCCTCGGTGCACATGCTTTACA
TGTGTTTAGTCGAGGTTAAAAAAACGTCTAGGCCCCCCGAACCACGGGGA
CGTGGTTTTCCTTTGAAAAACACGATGATAA.

173. A kit of claim 167, wherein the biotin ligase comprises a bacterial biotin ligase.

174. A kit molecule of claim 173, wherein the biotin ligase comprises a Bir A.

175. A kit of claim 167, wherein the eukaryotic promoter comprises a cytomegalovirus immediate early promoter.

176. A kit of claim 167, wherein the fusion protein further comprises an epitope tag.

177. A kit of claim 176, wherein the epitope tag is a FLAG sequence, a myc epitope tag, or a polyhistidine tag.

178. A kit of claim 167, wherein the site allowing for insertion of the DNA sequence is a restriction enzyme cleavage site.

179. A kit of claim 167, wherein the site allowing for insertion of the DNA sequence is a recombination site.

180. A kit of claim 167 wherein the site allowing for insertion of the DNA is a topoisomerase I-based site.

181. A kit of claim 167, which is double stranded DNA.

182. A kit of claim 167 wherein the DNA molecule is a circular DNA molecule.

183. A kit of claim 167 wherein the DNA molecule is a linear DNA molecule.

184. A kit of claim 167 wherein the DNA molecule comprises a vector.

185. A kit for generating a DNA molecule encoding a fusion protein comprising a selected protein and a biotinylation peptide, the kit comprising:
a DNA cassette comprising:
a) a DNA sequence encoding a biotinylation peptide;
b) a DNA sequence coding for an internal ribosome entry site; and
c) a DNA sequence encoding a biotin ligase;
wherein the DNA molecule is packaged in a container.

186. A kit of claim 185, wherein the fusion protein further comprises a protease cleavage site between the selected protein and the biotinylation peptide.

187. A kit of claim 185, wherein the fusion protein further comprises a leader peptide.

188. A kit of claim 185, wherein the biotinylation peptide comprises SEQ ID NO: 1 or a conservatively substituted variant thereof.

189. A kit of claim 185, wherein the internal ribosome entry site comprises a picornavirus internal ribosome entry site.

190. A kit of claim 189, wherein the picornavirus internal ribosome entry site comprises:
(SEQ ID NO: 4)
AATTCCGCCCCTCTCCCTCCCCCCCCCCTAACGTTACTGGCCGAAGCCGC
TTGGAATAAGGCCGGTGTGCGTTTGTCTATATGTGATTTTCCACCATATT
GCCGTCTTTTGGCAATGTGAGGGCCCGGAAACCTGGCCCTGTCTTCTTGA
CGAGCATTCCTAGGGGTCTTTCCCCTCTCGCCAAAGGAATGCAAGGTCTG
TTGAATGTCGTGAAGGAAGCAGTTCCTCTGGAAGCTTCTTGAAGACAAAC
AACGTCTGTAGCGACCCTTTGCAGGCAGCGGAACCCCCCACCTGGCGACA
GGTGCCTCTGCGGCCAAAGCCACGTGTATAAGATACACCTGCAAAGGCGG
CACAACCCCAGTGCCACGTTGTGAGTTGGATAGTTGTGGAAAGAGTCAAA
TGGCTCTCCTCAAGCGTATTCAACAAGGGGCTGAAGGATGCCCAGAAGGT
ACCCCATTGTATGGGATCTGATCTGGGGCCTCGGTGCACATGCTTTACAT
GTGTTTAGTCGAGGTTAAAAAAACGTCTAGGCCCCCCGAACCACGGGGAC
GTGGTTTTCCTTTGAAAAACACGATGATAA.

191. A kit of claim 185, wherein the biotin ligase comprises a bacterial biotin ligase.

192. A kit of claim 191, wherein the biotin ligase comprises a Bir A.

193. A kit of claim 185, wherein the eukaryotic promoter comprises a cytomegalovirus immediate early promoter.

194. A kit of claim 185, wherein the fusion protein further comprises an epitope tag.

195. A kit of claim 194, wherein the epitope tag is a FLAG sequence, a myc epitope tag, or a polyhistidine tag.

196. A kit of claim 185, wherein the DNA cassette is double stranded DNA.

197. A kit of claim 185, wherein the DNA cassette is an amplicon.

198. A kit of claim 185, wherein the DNA cassette is a linear DNA molecule.

199. A kit of claim 185, wherein the DNA cassette comprises a vector.

200. A kit for producing a biotinylated protein, the kit comprising:
an RNA molecule comprising:
a) an RNA sequence encoding a fusion protein comprising a selected protein and a biotinylation peptide; and
b) an RNA sequence encoding a biotin ligase operatively linked to an internal ribosome entry site;
wherein the RNA molecule is packaged in a container.

201. A kit of claim 200, wherein the wherein the fusion protein further comprises a protease cleavage site between the selected protein and the biotinylation peptide.

202. A kit of claim 200, wherein the fusion protein further comprises a leader peptide.

203. A kit of claim 200, wherein the biotinylation peptide comprises SEQ ID NO: 1 or a conservatively substituted variant thereof.

204. A kit of claim 200, wherein the internal ribosome entry site comprises a picornavirus internal ribosome entry site.

205. A kit of claim 204, wherein the picornavirus internal ribosome entry site comprises
(SEQ ID NO: 4)
AATTCCGCCCCTCTCCCTCCCCCCCCCCTAACGTTACTGGCCGAAGCCGC
TTGGAATAAGGCCGGTGTGCGTTTGTCTATATGTGATTTTCCACCATATT
GCCGTCTTTTGGCAATGTGAGGGCCCGGAAACCTGGCCCTGTCTTCTTGA
CGAGCATTCCTAGGGGTCTTTCCCCTCTCGCCAAAGGAATGCAAGGTCTG
TTGAATGTCGTGAAGGAAGCAGTTCCTCTGGAAGCTTCTTGAAGACAAAC
AACGTCTGTAGCGACCCTTTGCAGGCAGCGGAACCCCCCACCTGGCGACA
GGTGCCTCTGCGGCCAAAAGCCACGTGTATAAGATACACCTGCAAAGGCG
GCACAACCCCAGTGCCACGTTGTGAGTTGGATAGTTGTGGAAAGAGTCAA
ATGGCTCTCCTCAAGCGTATTCAACAAGGGGCTGAAGGATGCCCAGAAGG
TACCCCATTGTATGGGATCTGATCTGGGGCCTCGGTGCACATGCTTTACA
TGTGTTTAGTCGAGGTTAAAAAAACGTCTAGGCCCCCCGAACCACGGGGA
CGTGGTTTTCCTTTGAAAAACACGATGATAA.

206. A kit of claim 200, wherein the biotin ligase comprises a bacterial biotin ligase.

207. A kit of claim 206, wherein the biotin ligase comprises a Bir A.

208. A kit of claim 200, wherein the fusion protein further comprises an epitope tag.

209. A kit of claim 208, wherein the epitope tag is a FLAG sequence, a myc epitope tag, or a polyhistidine tag.

210. A kit of claim 200, wherein a host cell comprises the RNA molecule.

211. A kit of claim 210, wherein the host cell which is a mammalian cell.

212. A kit of claim 211, wherein the host cell is a COS cell.

213. A kit of claim 200, wherein the RNA molecule comprises an in vitro translation system.

214. A kit of claim 213, wherein the in vitro translation system is a reticulocyte lysate translation system, a wheat germ extract translation system, or a Xenopus oocyte extract translation system.
